(19) United States
(12) Patent Application Publication
(10) Pub. No.: US 2025/0369021 A1
(43) Pub. Date: Dec. 4, 2025

(54) GENETIC ENGINEERING OF ENDOGENOUS PROTEINS

(71) Applicant: The Regents of the University of California, Oakland, CA (US)

(72) Inventors: Theodore Lee Roth, San Francisco, CA (US); Po-Yi Jonanthan Li, San Francisco, CA (US); Alexander Marson, San Francisco, CA (US)

(21) Appl. No.: 19/096,426

(22) Filed: Mar. 31, 2025

Related U.S. Application Data
(63) Continuation of application No. 17/058,452, filed on Nov. 24, 2020, now abandoned.
(60) Provisional application No. 62/676,650, filed on May 25, 2018.
(60) Provisional application No. 62/818,367, filed on Mar. 14, 2019.

(51) Int. Cl. C12N 15/90; A61K 40/11; A61K 40/42; A61P 35/00; C07K 14/705; C07K 14/715; C07K 14/725; C12N 5/0783

(52) U.S. Cl. CPC C12N 15/907; A61K 40/11; A61K 40/4269; A61P 35/00; C07K 14/7051; C07K 14/70521; C07K 14/70578; C07K 14/715; C07K 14/7158; C12N 5/0637; C12N 2310/20; C12N 2510/00; C12N 2800/80

(57) ABSTRACT

Provided herein are methods and compositions for modifying an endogenous cell surface protein in a human cell by inserting a heterologous nucleic acid sequence in a target region of a nucleic acid encoding the endogenous cell surface protein.

### PRIOR RELATED APPLICATIONS

This application is a continuation of U.S. application Ser. No. 17/058,452, filed Nov. 24, 2020, which is a U.S. National Phase Application Under 35 U.S.C. § 371 of International Application No. PCT/US2019/033932, filed May 24, 2019, which claims the benefit of U.S. Provisional Application No. 62/676,650 filed on May 25, 2018 and U.S. Provisional Application No. 62/818,367, filed on Mar. 14, 2019, which applications are hereby incorporated by reference in their entireties for all purposes.

### SEQUENCE LISTING

The instant application contains a Sequence Listing which has been submitted electronically in ASCII format and is hereby incorporated by reference in its entirety. Said ASCII copy, created on Nov. 20, 2020, is named 081906-1220009-228520US SL.txt and is 21,714 bytes in size.

### BACKGROUND OF THE INVENTION

Current techniques for modification of ex vivo or intravitally gene edited cells for therapeutic use have focused on correction of an existing mutation, limiting therapeutic applicability to conditions caused by a single mutation resulting in a misfunctioning gene, or on integrating an entirely new synthetic gene, requiring extensive research and development into creating a new therapeutically useful synthetic DNA sequence. Therefore, there are limited options for endogenous gene modifications. Given the importance of T cells in adoptive cellular therapeutics, the ability to obtain human T cells and modify their endogenous proteins to produce edited T cells with desirable function(s) could be beneficial in the development and application of adoptive T cell therapies.

### BRIEF SUMMARY OF THE INVENTION

The present disclosure is directed to compositions and methods for modifying the genome of a human cell, for example, a T cell. The inventors have discovered that endogenous genes encoding cell surface proteins in human T cells can be modified to alter the functionality of the endogenous cell surface protein in the human T cell. For example, a functional domain can be added to an endogenous cell surface receptor to enhance a favorable activity, for example signaling activity by the endogenous cell surface receptor. By inserting a nucleic acid encoding a functional domain into the endogenous gene encoding a cell surface protein, human T cells comprising one or more modified endogenous cell surface proteins, that take advantage of already existing regulatory and signaling pathways in the T cell, can be made. Further, the compositions and methods described herein can be used to generate human T cells with altered functionality, while limiting the side effects associated with T cell therapies.

The inventors have further discovered that coding sequences for individual (e.g., not as a domain fusion to an endogenous protein) heterologous proteins can be added to an endogenous gene sequence thereby allowing for co-regulation of the heterologous protein by the endogenous gene control sequences. The heterologous protein can be co-expressed with the endogenous protein or instead of the endogenous protein as explained below.

The methods and compositions provided herein can be used to modify an endogenous cell surface protein in a human T cell or other human cell by inserting a heterologous nucleic acid sequence encoding a functional domain in a target region of a nucleic acid encoding the endogenous cell surface receptor. In some embodiments, the target region in the genome of a T cell is a native or endogenous protein locus, for example, a native or endogenous cell surface protein locus. In some examples, the native or endogenous protein locus, is a native or endogenous cell surface receptor protein locus.

Provided herein is a method of modifying an endogenous cell surface protein in a human T cell. In some embodiments, the method comprises (a) introducing into the human T cell, (i) a targeted nuclease that cleaves a target region in a nucleic acid encoding the endogenous cell surface protein to create an insertion site in the genome of the cell; and (ii) a heterologous nucleic acid sequence encoding a functional domain or a functional fragment thereof, wherein the nucleic acid sequence is flanked by homologous sequences, and (b) allowing homologous recombination to take place, thereby inserting the nucleic acid sequence in the insertion site to generate a modified human T cell comprising a modified endogenous cell surface protein, wherein the heterologous functional domain or functional fragment thereof is linked to the cytoplasmic domain of the endogenous cell surface protein, and wherein the modified endogenous cell surface protein of the T cell has the activity of the heterologous functional domain or a functional fragment thereof.

In some embodiments, the modified endogenous cell surface protein has a binding specificity of the endogenous cell surface protein and an activity of the functional domain or a functional fragment thereof. In some embodiments, the activity of the functional domain or a functional fragment thereof is signaling activity. In some embodiments, the targeted nuclease cleaves a target region in an exon encoding the N-terminus of the endogenous cell surface protein or a target region in an exon encoding the C-terminus of the endogenous cell surface protein.

In some embodiments, the targeted nuclease cleaves a target region in an exon encoding the N-terminus of the endogenous cell surface protein; and the nucleic acid sequence encodes in the following order, (1) a selectable marker; (2) a self-cleaving peptide sequence; and (3) the functional domain or a functional fragment thereof.

In some embodiments, the targeted nuclease cleaves a target region in an exon encoding the C-terminus of the endogenous cell surface protein; and wherein the nucleic acid sequence encodes in the following order, (1) the functional domain or a functional fragment thereof; (2) a self-cleaving peptide sequence; and (3) a selectable marker.

In some embodiments, the targeted nuclease cleaves a target region in an exon encoding the C-terminus of the cell surface protein and the functional domain is a cytoplasmic domain of an intracellular signaling protein or a functional fragment thereof.

In some embodiments, the modified endogenous cell surface protein of the T cell has a binding specificity of the endogenous cell surface protein and the signaling activity of the cytoplasmic domain of the intracellular signaling protein or a functional fragment thereof.

In some embodiments, the endogenous cell surface protein is selected from the group consisting of a T cell receptor (TCR) complex protein, a co-stimulatory receptor, a co-inhibitory receptor, a cytokine receptor and a chemokine receptor. In some embodiments, the TCR complex protein is selected from the group consisting of: the TCR-α chain, the TCR-β chain, the CD3δ chain, the CD3ε chain, the CD3γ chain, and the CD3ζ chain of the endogenous TCR complex.

In some embodiments, the TCR complex of the T cell comprises the modified endogenous TCR complex protein, and the TCR complex of the T cell has the antigen-binding specificity of the endogenous TCR and the signaling activity of the cytoplasmic domain of the intracellular signaling protein or a functional fragment thereof.

In some embodiments, the cytoplasmic domain of the intracellular signaling protein is the cytoplasmic domain of a co-stimulatory receptor or a functional fragment thereof. In some embodiments, the cytoplasmic domain of the intracellular signaling protein is the cytoplasmic domain of an adaptor protein or a functional fragment thereof. In some embodiments, the co-stimulatory receptor is CD28 or 41BB. In some embodiments, the adaptor protein is DAP10 or MYD88.

In some embodiments, one or more TCR complex proteins are modified by inserting the heterologous nucleic acid sequence into an exon encoding the C-terminus of an endogenous TCR complex protein. In some embodiments, the TCR complex comprises one or more modified endogenous TCR complex proteins linked to the cytoplasmic domain of a co-stimulatory receptor or a functional fragment thereof. In some embodiments, the heterologous nucleic acid sequence encoding the cytoplasmic domain of the co-stimulatory receptor or a functional fragment thereof is inserted downstream of the last amino acid of the endogenous TCR complex protein and upstream of the stop codon for the endogenous TCR complex protein.

Also provided is a method of modifying a human T cell, the method comprising: (a) introducing into the human T cell (i) a targeted nuclease that cleaves a target region in exon 1 of a TCR-alpha subunit constant gene (TRAC) in the human T cell to create an insertion site in the genome of the cell; (ii) a heterologous nucleic acid sequence encoding, in the following order, (1) a first self-cleaving peptide sequence; (2) a full-length T cell receptor (TCR)-β chain; (3) the cytoplasmic domain of a co-stimulatory receptor or a functional fragment thereof; (4) a second self-cleaving peptide sequence; (5) a variable region of a TCR-α chain; and (6) a portion of the N-terminus of the endogenous TCR-α chain, wherein the nucleic acid sequence is flanked by homologous sequences; and (b) allowing recombination to occur, thereby inserting the nucleic acid sequence in the insertion site to generate a modified human T cell, wherein the heterologous cytoplasmic domain of the co-stimulatory receptor or a functional fragment thereof is linked to the cytoplasmic domain of the full-length T cell receptor (TCR)-β chain, and wherein the modified TCR complex of the T cell is antigen-specific and has the signaling activity of the cytoplasmic domain of the co-stimulatory receptor or a functional fragment thereof.

In some embodiments, the nucleic acid encodes a full-length endogenous T cell receptor (TCR)-β chain linked to the cytoplasmic domain of co-stimulatory receptor or a functional fragment thereof and the variable region of an endogenous TCR-α chain. In some embodiments, the nucleic acid encodes a full-length heterologous T cell receptor (TCR)-β chain linked to the cytoplasmic domain of co-stimulatory receptor or a functional fragment thereof and a variable region of a heterologous TCR-α chain. In some embodiments, the co-stimulatory receptor is CD28 or 41BB.

In some embodiments, the targeted nuclease introduces a double-stranded break at the insertion site. In some embodiments, the targeted nuclease is an RNA-guided nuclease. In some embodiments, the RNA-guided nuclease is a Cpf1 nuclease or a Cas9 nuclease and the method further comprises introducing into the cell a guide RNA that specifically hybridizes to the target region. In some embodiments, the Cpf1 nuclease or the Cas9 nuclease, the guide RNA and the nucleic acid are introduced into the cell as a ribonucleoprotein complex (RNP)-nucleic acid sequence complex, wherein the RNP-nucleic acid sequence complex comprises: (i) the RNP, wherein the RNP comprises the Cpf1 nuclease or the Cas9 nuclease and the guide RNA; and (ii) the nucleic acid sequence.

In some embodiments, the T cell is a primary T cell. In some embodiments, the primary T cell is a regulatory T cell. In some embodiments, the primary T cell is a CD8+ T cell or a CD4+ T cell. In some embodiments, the primary T cell is a CD4+CD8+ T cell.

In some embodiments, the method further comprises culturing the modified T cells under conditions effective for expanding the population of modified cells. In some embodiments, the method further comprises purifying T cells that express the modified endogenous cell surface protein. Also provided are modified human T cells produced by any of the methods provided herein.

Further provided is a method of enhancing an immune response in a human subject comprising: a) obtaining T cells from the subject; b) modifying the T cells to express an antigen-specific TCR complex that recognizes a target antigen in the subject using any of the methods provided herein; and c) administering the modified T cells comprising the modified TCR complex to the subject. In some embodiments, the human subject has cancer and the target antigen is a cancer-specific antigen. In some embodiments, the human subject has an autoimmune disorder and the antigen is an antigen associated with the autoimmune disorder. In some embodiments, the T cells are regulatory T cells. In some embodiments, the subject has an infection and the target antigen is an antigen associated with the infection.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a schematic depicting insertion of a non-viral DNA template comprising a nucleic acid sequence encoding, in the following order, a co-stimulatory cytoplasmic domain; a P2A self-cleaving peptide; and an optional selectable marker, i.e. GFP, into a T cell via homology directed repair.

FIG. 2 is a schematic depicting insertion of a non-viral DNA template comprising a nucleic acid sequence encoding, in the following order, (i) a T2A self-cleaving peptide sequence; (ii) a full-length heterologous TCR-β chain (NYESO-β); (iii) a cytoplasmic domain of a co-stimulatory receptor (iv) a P2A self-cleaving peptide sequence; (v) a variable region of a heterologous TCR-α chain (NYESO-α); and (v) a portion of the N-terminus of the endogenous TCR alpha subunit into a T cell via homology directed repair. After insertion of the DNA template in exon 1 of the TRAC gene via homology directed repair, the DNA template was transcribed and translated to produce a full-length NYESO-β chain comprising the cytoplasmic domain of a co-stimulatory receptor and a full-length NYESO-α chain that forms an antigen-specific TCR that recognizes the NY-ESO-1 melanoma neoantigen.

FIG. 3 illustrates an exemplary T cell therapy pipeline using T cells with modified endogenous proteins.

FIG. 4 depicts flow cytometry results showing that individual protein members of the T cell receptor complex can be tagged with a fluorescent selectable marker.

FIG. 5 depicts flow cytometry results showing that, in addition to tagging individual components of the TCR complex with a fluorescence marker, it was possible to multiplex and tag multiple components of the TCR complex with multiple fluorescence markers simultaneously.

FIG. 6 shows the in vitro cancer cell killing efficacy of NY-ESO-1 specific T-cells compared to NY-ESO-1 specific T-cells where the TCR has been tagged with either the cytoplasmic domain of 41BB or CD28. Cancer cell killing was analyzed using the IncuCyte platform, which captures an image of each sample and determines a count of fluorescent cells by image analysis. The in vitro killing assay utilized a cancer cell line that expresses both red fluorescent protein (RFP) and the NY-ESO-1 melanoma neoantigen as the target. Samples seeded with T cells only, which do not express any fluorescent proteins, saw no measurement of cell growth by IncuCyte, whereas samples seeded with only cancer cells saw a sharp increase in cell count followed by a plateau, as the well's carrying capacity was reached. In all four samples where cancer cells were co-cultured with NY-ESO-1 TCR+ T cells, cancer cell killing was observable, but the NY-ESO-1 specific T cells modified with co-stimulatory cytoplasmic domains killed cancer cells better.

FIG. 7 shows the expression patterns of representative inhibitory and activating cell surface markers. The T cells analyzed were recovered from the in vitro cancer cell killing assay and profiled by flow cytometry. NYESO-CD28 and NYESO-41BB T cells had lower PD1 and CD25 cell surface expression levels at the end of the killing assay.

FIGS. 8A-G: Genetically Engineered Endogenous Proteins (GEEPs)

FIG. 8A, Schematic description of all the different ways that were validated for engineering cell-surface proteins at the endogenous gene locus. Within any given cell-surface protein's gene locus, we can modify (from left to right) the 5′ non-coding region to override endogenous gene regulation with a synthetic/exogenous promoter, add or replace the protein expressed under a particular endogenous promoter, replace receptor specificity by targeting a sequence encoding a novel extracellular domain to the exon encoding the transmembrane domain, and alter the signaling of a receptor by knocking-in new signaling domain(s).

FIG. 8B, To test whether we could tune gene expression by knocking-in a synthetic promoter, we targeted a SFFV promoter to the 5′ non-coding region of IL2RA and PDCD1. When we analyzed edited T cells cultured without restimulation by flow cytometry 7 days after electroporation, we saw that successful knock-in led to sustained expression of either protein. (top). We show that T cells edited with on-target conditions for IL2RA (IL2RA RNP+SFFV HDR DNA Template) maintain high expression of CD25 whereas T cells edited with control conditions (Scrambled RNP+SFFV HDR DNA Template) see CD25 expression levels return to baseline. (Bottom) Similarly, T cells edited with on-target conditions for PDCD1 (PDCD1 RNP+SFFV HDR DNA Template) maintain high levels of PD1 whereas T cells edited with control conditions see PD1 expression levels return to baseline.

FIG. 8C, To test whether we could put a synthetic product under the regulation of an endogenous promoter, we targeted an insert encoding tNGFR and either a 2A sequence or a Poly A tail to the N-terminal coding region of PD1 such that tNGFR would be expressed with or without PD1, respectively, under the regulation of the PD1 promoter. When we restimulated edited T cells and analyzed them by flow cytometry 48 hours later, we saw high co-expression of PD1 and tNGFR with the tNGFR-2A insert (Top) and high expression of tNGFR along with PD1 KO with the tNGFR-Poly A insert (Bottom).

FIG. 8D, To test whether we could alter the extracellular specificity of a receptor, we tested to see whether we could alter TCR specificity. Using a previously described targeting strategy, we were able to knock-in the 1G4 TCR receptor into the endogenous TRAC locus with a very high knock-in efficiency.

FIG. 8E, To test whether we could knock-in additional or replacement signaling domains to create synthetic signaling cascades, we designed constructs that would incorporate either the CD28 or 41BB intracellular domain on the C-terminus of one of the CD3 subunits. To make readout of an intracellular domain knock-in easier, we also included a fluorescent protein preceded by a 2A sequence in the construct as a marker for successful knock-in. Successful integration would yield a polycistronic sequence expressing a CD3 chain containing a new signaling domain fused to the C-terminus and a fluorescent protein simultaneously. (Top) We show successful integration of the CD28 intracellular domain at the C-terminus of CD3 epsilon, as measured by the percentage of GFP+ cells. (Bottom) Additionally, we show successful integration of the 41BB intracellular domain at the C-terminus of CD3 epsilon, as measured by the percentage of mCherry+ cells.

FIG. 8F, To test whether putting a synthetic product under an endogenous promoter truly mimicked the corresponding endogenous protein's expression dynamics, we profiled T cells with tNGFR-2A knocked in to the IL2RA N terminus by flow cytometry over the course of 5 days and compared tNGFR expression dynamics to that of IL2RA. In both CD8 and CD4 subsets, IL2RA and tNGFR expression both decreased over time in the absence of restimulation. Similarly, in restimulated cells, both CD8 and CD4 cells saw a simultaneous upregulation of IL2RA and tNGFR.

FIG. 8G, Results of a competitive mixed proliferation assay testing the advantage of synthetic CD3 signaling. We pooled unsorted edited T cells with CD28IC-2A-GFP, 41BBIC-2A-mCherry, or 2A-BFP knocked-in to the same CD3 complex member's gene locus. We then cultured the mixed cell population without stimulation, with CD3 stimulation only, with CD28 stimulation only, or with CD3/CD28 stimulation. After 4 days in culture, samples were analyzed by flow cytometry for relative outgrowth of GFP+ and mCherry+ subpopulations relative to the BFP+ subpopulation. We then normalized the proportions to those found in the corresponding unstimulated condition.

FIGS. 9A-D: Genetically Engineered Endogenous Proteins with synthetic regulation of endogenous products.

FIG. 9A, Schematic describing our knock-in strategy for targeting a novel promoter to the N-terminus of a gene of interest with or without an additional selection marker.

FIG. 9B, Representative flow data for our knock-in strategy wherein we integrate (in 5′ to 3′ order) a SFFV promoter, a selection marker tNGFR, and a 2A sequence such that a multicistronic mRNA that produces two proteins, tNGFR and the endogenous protein, is being expressed off an SFFV promoter at defined endogenous gene locus. We targeted the N-terminus of three immune receptors, PD1, LAG3, and IL2RA, whose expression are highly upregulated upon T-cell activation. In the top row, we observe that expression levels of each respective immune receptor in cells that have been cultured for 7 days post electroporation without restimulation. Consistently, we observe that in control conditions (Scrambled RNP+HDR DNA Template) expression levels or immune receptor are relatively low. In the on target conditions (On-target RNP+HDR DNA Template), we see that tNGFR+ cells, which also have the SFFV promoter knocked in, have high levels of expression of each of the immune receptors while the tNGFR-cells have expression levels similar or lower than the control, the latter most likely attributed to KO occurring with the on-target RNP in the absence of HDR DNA Template integration. When we restimulated these cells, we see that the expression levels of each of the immune receptors increase in the control samples. In the restimulated on-target samples, the tNGFR+ cells retain high expression levels of each respective immune receptor whereas the tNGFR-cells upregulate expression levels, although to a lesser extent.

FIG. 9C, When we compare tNGFR expression levels against expression levels of the respective immune receptor in control and on-Target edited cells that have not been restimulated, we see that on-target cells have high expression levels of both tNGFR and their respective immune receptor (demonstrated by the linear relationship) while the control cells have lower expression levels of the respective immune receptor and negligible tNGFR expression.

FIG. 9D, Having validated our knock-in strategy for integrating a novel/synthetic promoter along with a selection marker, we applied our knock-in strategy to an array of transcription factors whose overexpression may be beneficial for T-cell proliferation and long-term function. To readout successful integration of our construct, we examined tNGFR expression levels in on-target samples for four different transcription factors and found that we were able to achieve 10-25% knock-in efficiency. This strategy has implications for being able to efficiently modulate transcription factor expression and subsequent T-cell function.

FIGS. 10A-E: Creation of Genetically Engineered Endogenous Proteins with endogenous regulation of synthetic products at PDCD1 locus.

FIG. 10A, Schematic describing our knock-in strategy for targeting novel protein(s) to the N-terminus of a gene of interest for coordinated expression of the novel protein(s) and the endogenous protein or expression of the novel protein(s) with knock out of the endogenous protein under endogenous gene regulation.

FIG. 10B, Representative flow plots validating our strategy for coordinated expression of a novel protein and PD1 under the endogenous gene regulation of PD1. In rested cells (top row), there is minimal PD1 and tNGFR expression. However, by 48 hours after restimulation with CD3/CD28 Dynabeads, we see a coordinated upregulation of tNGFR and PD1.

FIG. 10C, Representative flow plots validating our strategy for simultaneous expression of a novel protein and knock out of PD1 under the endogenous gene regulation of PD1. In rested cells (top row), there is minimal PD1 and tNGFR expression. However, by 48 hours after restimulation with CD3/CD28 Dynabeads, we see upregulation of tNGFR and without upregulation of PD1.

FIG. 10D, Representative flow plots validating our strategy for coordinated expression of multiple novel proteins and PD1 under the endogenous gene regulation of PD1. Based on tNGFR readout, we were able to successfully integrate our novel construct at the PDCD1 gene locus.

FIG. 10E, Representative flow plots validating our strategy for simultaneous expression of multiple novel proteins and knock-out of PD1 under the endogenous gene regulation of PD1. Based on tNGFR readout, we were able to successfully integrate our novel construct at the PDCD1 gene locus.

FIGS. 11A-D: Genetically Engineered Endogenous Proteins with endogenous regulation of synthetic products.

FIG. 11A, Schematic describing our knock-in strategy for targeting a novel protein to the N-terminus of a gene of interest for coordinated expression of the novel protein and the endogenous protein under endogenous gene regulation

FIG. 11B, Representative flow data from experiments wherein we integrate a tNGFR-2A construct at the N-terminus of IL2RA. We demonstrate tNGFR expression levels differ depending integration site, time, and cell culture conditions and, importantly, mirror that of that of the endogenous protein whose promoter is controlling expression. In cells where the target site was IL2RA, we see a linear IL2RA high, tNGFR high population at Day 3 post-electroporation, indicative of coordinated expression of the two. At Day 7 post-electroporation, cells that were cultured without restimulation see a gradual and coordinated decreased expression of both IL2RA and tNGFR whereas in cells that were restimulated, we see the maintenance of an IL2RA high, tNGFR high population.

FIG. 11C, Representative flow data from experiments wherein we integrate a tNGFR-2A construct at the N-terminus of CD28. We similarly observe a linear CD28 high tNGFR high population at Day 3. CD28 expression levels remain high without restimulation and that is reflected in our Day 7 analyses. In cells that were cultured without restimulation, we see a sustained CD28 high tNGFR high population where as in restimulated cells, we see a simultaneous modulation of CD28 and tNGFR expression. The more drastic decrease of CD28 expression could be due to the combination of gene expression modulation and internalization of the protein whereas tNGFR is not being internalized.

FIG. 11D, Representative flow data from experiments wherein we integrate a tNGFR-2A construct at the N-terminus of Lag3. At Day 3, Lag3 and tNGFR expression were negligible and both levels of expression remained low without restimulation at Day 7. However, when we restimulated the cells and analyzed them on Day 7, we saw the simultaneous upregulation of Lag3 and tNGFR.

FIGS. 12A-B: Creation of Genetically Engineered Endogenous Proteins with endogenous specificity and synthetic signaling in CD3 complex members.

FIG. 12A, Schematic describing the three different constructs we designed to modify the C-terminus of each of the different CD3 subunits in the TCR complex, which include the CD3δ chain, CD3ε chain, CD3γ chain, and CD3ζ chain. For initial tests, we designed a construct that would knock-in a 2A-BFP at the C-terminus of each of the different CD3 subunits. The 2A-BFP integration would create a multicistronic mRNA that produces two separate proteins: an unmodified CD3 chain and BFP. Once the 2A-BFP integration was validated, we modified the construct to include a cytoplasmic domain of an activating immune receptor before the 2A sequence such that the C-terminus of the CD3 subunit chain now contains an additional signaling domain/motif.

FIG. 12B, To readout successful integration of the signaling domain, we analyzed the percentage of fluorescent protein expressing T-cells by flow cytometry. The addition of an extra signaling domain did not have a significant/consistent effect on knock-in efficiency. The positioning of the additional signaling domain relative to endogenous CD3 signaling motifs was not optimized, but the ability to modify the intracellular domains of individual CD3 subunits provides a promising platform for tuning TCR signaling.

### DEFINITIONS

As used in this specification and the appended claims, the singular forms “a,” “an,” and “the” include plural reference unless the context clearly dictates otherwise.

The term “nucleic acid” or “nucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (e.g., a single guide RNA), or micro RNA.

As used herein, the term “endogenous” with reference to a nucleic acid, for example, a gene, or a protein in a cell is a nucleic acid or protein that occurs in that particular cell as it is found in nature, for example, at its natural genomic location or locus. Moreover, a cell “endogenously expressing” a nucleic acid or protein expresses that nucleic acid or protein as it is found in nature.

The term “functional domain” refers to a part of a protein sequence that can function independently of the protein sequence from which it is derived, for example, when incorporated into or attached to a different protein sequence. When linked to or inserted into a protein, for example, an endogenous cell surface protein of a T cell, the functional domain retains one or more activities normally associated with the functional domain when it is part of the protein sequence from which it is derived. Therefore, the endogenous cell surface protein acquires one or more activities normally associated with the functional domain. The degree or amount of an activity of the functional domain, once linked to or inserted into the endogenous cell surface protein can vary as compared to the degree or amount of an activity of the functional domain when it is part of its native protein. For example, the degree or amount of an activity of a functional domain inserted into or linked to an endogenous cell surface protein of a T cell can be at least 50%, 60%, 70%, 80%, 90% or greater than the degree or amount of an activity of the functional domain when it is part of its native protein. These activities include, but are not limited to, signaling activity, binding activity, enzymatic activity, transcriptional regulatory activity and dimerization activity. A functional domain can also be a synthetic domain designed to improve one or more properties of an endogenous cell surface protein, and need not be a naturally occurring amino acid sequence. For example, the amino acid sequence of a naturally occurring functional domain that has been modified to improve one or more properties of the functional domain can be linked to or inserted into an endogenous protein. Therefore, an amino acid sequence having at least 70%, 80% or 90% identity with a naturally amino acid sequence of a functional domain, that retains one or more activities of the functional domain, can also be used as a functional domain. The functional domain can be linked to the C-terminus or the N-terminus of the endogenous cell surface protein. For example, the functional domain can be linked to the C-terminus of an endogenous cell surface protein immediately after the last amino acid of the endogenous protein sequence. In another example, the functional domain can be linked to the N-terminus of an endogenous cell surface protein immediately prior to the first amino acid of the endogenous protein sequence. The functional domain can also be inserted into an internal amino acid sequence of the endogenous cell surface protein. For example, the functional domain can be inserted before or after the transmembrane domain to replace the extracellular domain or intracellular domain, respectively, of the endogenous cell surface protein. The functional domain or functional fragment thereof can be at least about 10, 20, 30, 40, 50, 60, 70, 80, 90, 100, 120 or 150 amino acids in length, as long as the functional domain or functional fragment thereof, when linked to or inserted into the endogenous cell surface protein, has one or more activities normally associated with the functional domain. In some cases, the functional domain is a cytoplasmic domain of an intracellular signaling protein or a functional fragment thereof. As used herein, “an intracellular signaling protein” is a protein involved in transmission of signals across the cell membrane.

As used herein, the term “selectable marker” refers to a gene which allows selection of a host cell, for example, a T cell, comprising a marker. The selectable markers may include, but are not limited to: fluorescent markers, luminescent markers and drug selectable markers, cell surface receptors, and the like. In some embodiments, the selectable marker is a non-immunogenic receptor, for example, a truncated receptor. In some embodiments, the selection can be positive selection; that is, the cells expressing the marker are isolated from a population, e.g. to create an enriched population of cells expressing the selectable marker. Separation can be by any convenient separation technique appropriate for the selectable marker used. For example, if a fluorescent marker is used, cells can be separated by fluorescence activated cell sorting, whereas if a cell surface marker has been inserted, cells can be separated from the heterogeneous population by affinity separation techniques, e.g. magnetic separation, affinity chromatography, “panning” with an affinity reagent attached to a solid matrix, fluorescence activated cell sorting or other convenient technique.

As used herein “a TCR complex” is a complex comprising a TCR-α chain, a TCR-β chain and three signaling dimers, CD3δ/ε, CD3γ/ε and CD3 ζ/ζ. CD3 ζ is also known as CD247 ζ. Ionizable residues in the transmembrane domain of each member of the TCR complex form a polar network of interactions that hold the complex together. The T cell receptor (TCR) of the TCR complex is a heterodimer comprising the TCR-α chain and the TCR-β chain and determines the antigen binding specificity of the TCR complex. Once the TCR of a TCR complex engages with antigenic peptide and MHC (peptide/MHC), the T lymphocyte is activated through signal transduction, primarily mediated through one or more of the CD3 chains of the complex and other signaling molecules such as, but not limited to, co-stimulatory receptors and/or co-inhibitory receptors. Examples of co-stimulatory receptors include, but are not limited to, CD28, ICOS and 41BB. Examples of co-inhibitory receptors include, but are not limited to, PD-1, LAG3, TIM-3 and CTLA-4. Adaptor proteins may also be used, for example, DAP10 or MYD88.

As used herein “a TCR complex protein” is a protein that is a protein member or component of a TCR complex in a T cell. Protein members of the TCR complex include a TCR-α chain, a TCR-β chain, a CD3δ chain, a CD3ε chain, a CD3γ chain, and a CD3ζ chain of a TCR complex.

“Treating” refers to any indicia of success in the treatment or amelioration or prevention of the disease, condition, or disorder, including any objective or subjective parameter such as abatement; remission; diminishing of symptoms or making the disease condition more tolerable to the patient; slowing in the rate of degeneration or decline; or making the final point of degeneration less debilitating.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequences that are perfectly complementary or substantially complementary (e.g., having 1-4 mismatches) to a genomic sequence.

As used throughout, by subject is meant an individual. For example, the subject is a mammal, such as a primate, and, more specifically, a human. The term does not denote a particular age or sex. Thus, adult and newborn subjects, whether male or female, are intended to be covered. As used herein, patient or subject may be used interchangeably and can refer to a subject afflicted with a disease or disorder.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39): 15644-9; Sampson et al., Nature. 2013 May 9; 497(7448): 254-7; and Jinek, et al., Science. 2012 Aug. 17; 337(6096): 816-21. Variants of any of the Cas9 nucleases provided herein can be optimized for efficient activity or enhanced stability in the host cell. Thus, engineered Cas9 nucleases are also contemplated. See, for example, “Slaymaker et al., “Rationally engineered Cas9 nucleases with improved specificity,” Science 351 (6268): 84-88 (2016)).

As used herein, the term “Cas9” refers to an RNA-mediated nuclease (e.g., of bacterial or archeal origin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p 759-771, 22 Oct. 2015) and homologs thereof. Similarly, as used herein, the term “Cas9 ribonucleoprotein” complex and the like refers to a complex between the Cas9 protein, and a crRNA (e.g., guide RNA or single guide RNA), the Cas9 protein and a trans-activating crRNA (tracrRNA), the Cas9 protein and a guide RNA, or a combination thereof (e.g., a complex containing the Cas9 protein, a tracrRNA, and a crRNA guide RNA). It is understood that in any of the embodiments described herein, a Cas9 nuclease can be substituted with a Cpf1 nuclease or any other guided nuclease.

As used herein, the phrase “modifying” in the context of modifying a genome of a cell refers to inducing a structural change in the sequence of the genome at a target genomic region. For example, the modifying can take the form of inserting a nucleotide sequence into the genome of the cell. For example, a nucleotide sequence encoding a polypeptide can be inserted into the genomic sequence encoding an endogenous cell surface protein in the T cell. The nucleotide sequence can encode a functional domain or a functional fragment thereof. Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA, or pair of guide RNAs, directed to the target genomic region.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein the phrase “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

As used herein, a “cell” refers to a human cell that expresses an endogenous cell surface protein, for example, a human T cell or a cell capable of differentiating into a T cell that expresses a TCR receptor molecule. These include hematopoietic stem cells and cells derived from hematopoietic stem cells.

As used herein, the phrase “hematopoietic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117⁺, lin⁻. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy1^{+/lo}, CD38⁺, C-kit⁺, lin⁻. In some cases, the hematopoietic stem cells are CD150⁺CD48⁻CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoietic cell obtained or provided by differentiating the stem cell. Hematopoietic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. In some embodiments, the hematopoietic cell is an immune cell, such as a T cell, B cell, macrophage, a natural killer (NK) cell or dendritic cell. In some embodiments the cell is an innate immune cell.

As used herein, the phrase “T cell” refers to a lymphoid cell that expresses a T cell receptor molecule. T cells include human alpha beta (αβ) T cells and human gamma delta (78) T cells. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4⁺, CD8⁺, or CD4⁺ and CD8⁺. T cells can also be CD4⁻, CD8⁻, or CD4⁻ and CD8⁻ T cells can be helper cells, for example helper cells of type T_{H}1, T_{H}2, T_{H}3, T_{H}9, T_{H}17, or T_{FH}. T cells can be cytotoxic T cells. Regulatory T cells can be FOXP3⁺ or FOXP3⁻. T cells can be alpha/beta T cells or gamma/delta T cells. In some cases, the T cell is a CD4⁺CD25^{hi}CD127^{lo} regulatory T cell. In some cases, the T cell is a regulatory T cell selected from the group consisting of type 1 regulatory (Tr1), T_{H}3, CD8+CD28−, Treg17, and Qa-1 restricted T cells, or a combination or sub-population thereof. In some cases, the T cell is a FOXP3⁺ T cell. In some cases, the T cell is a CD4⁺CD25^{lo}CD127^{hi} effector T cell. In some cases, the T cell is a CD4⁺CD25^{lo}CD127^{hi}CD45RA^{hi}CD45RO⁻ naïve T cell. A T cell can be a recombinant T cell that has been genetically manipulated.

As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. The homologous template nucleic acid can be provided by homologous sequences elsewhere in the genome (sister chromatids, homologous chromosomes, or repeated regions on the same or different chromosomes). Alternatively, an exogenous template nucleic acid can be introduced to obtain a specific HDR-induced change of the sequence at the target site. In this way, specific mutations can be introduced at the cut site.

As used herein, a single-stranded DNA template or a double-stranded DNA template refers to a DNA oligonucleotide that can be used by a cell as a template for editing or modifying the genome of T cell, for example, by HDR. Generally, the single-stranded DNA template or a double-stranded DNA template has at least one region of homology to a target site. In some cases, the single-stranded DNA template or double-stranded DNA template has two homologous regions, for example, a 5′ end and a 3′ end, flanking a region that contains a heterologous sequence to be inserted at a target cut or insertion site.

The term “substantial identity” or “substantially identical,” as used in the context of polynucleotide or polypeptide sequences, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

### DETAILED DESCRIPTION OF THE INVENTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

The present disclosure is directed to compositions and methods for modifying the genome of a human cell. The inventors have discovered that endogenous genes encoding cell surface proteins in human T cells can be modified to alter the functionality of the endogenous cell surface protein in the human T cell. By inserting a heterologous nucleic acid encoding a functional domain into the endogenous gene encoding a cell surface protein, human T cells comprising one or more modified endogenous cell surface proteins having the activity of the functional domain can be made. These modified T cells can be used, for example, to treat cancer, autoimmune disease or infection in a subject.

In some embodiments, a heterologous nucleic acid encoding a functional domain or a functional fragment thereof is inserted into an exon of a gene encoding the C-terminus of an endogenous cell surface protein or an exon of a gene encoding the N-terminus of an endogenous cell surface protein in the genome of the T cell to produce a modified T cell comprising a modified endogenous cell surface protein having the activity of the functional domain.

In some embodiments, a heterologous nucleic acid encoding the cytoplasmic domain of a co-stimulatory receptor or a functional fragment thereof, is inserted into an exon of a gene encoding the C-terminus of a gene encoding a TCR complex protein in the genome of the T cell to produce a modified T cell comprising a modified TCR complex having the antigen binding specificity of the TCR and the signaling activity of the cytoplasmic domain of the co-stimulatory receptor. In some embodiments, the signaling activity of the TCR complex increases by at least 10%, 20%, 30%, 50%, 60%, 70%, 80%, 90%, 100%, 200%, 300%, 400% or greater as compared to the signaling activity of the endogenous or non-modified TCR complex of the T cell. Downstream activities after antigen binding and activation of the T cell, for example, cancer cell killing, as described in the Examples, can be measured to determine the signaling activity of the T cell comprising the modified TCR complex.

In some embodiments, a heterologous nucleic acid sequence encoding a full-length TCR-β chain, the cytoplasmic domain of a co-stimulatory receptor or a functional fragment thereof and a variable region of a heterologous TCR-α chain is inserted into exon 1 of the TRAC gene in the genome of the human T cell to produce a modified human T cell. In these modified T cells, the heterologous cytoplasmic domain of the co-stimulatory receptor or a functional fragment thereof is linked to the cytoplasmic domain of the full-length T cell receptor (TCR)-β chain to produce a modified TCR that is antigen-specific and has the signaling activity of the cytoplasmic domain of the co-stimulatory receptor.

In some embodiments, a heterologous nucleic acid sequence encoding a full-length TCR-α chain, the cytoplasmic domain of a co-stimulatory receptor or a functional fragment thereof and a variable region of a heterologous TCR-β chain is inserted into exon 1 of the TRBC gene in the genome of the human T cell to produce a modified human T cell. In these modified T cells, the heterologous cytoplasmic domain of the co-stimulatory receptor or a functional fragment thereof is linked to the cytoplasmic domain of the full-length T cell receptor (TCR)-α chain to produce a modified TCR that is antigen-specific and has the signaling activity of the cytoplasmic domain of the co-stimulatory receptor.

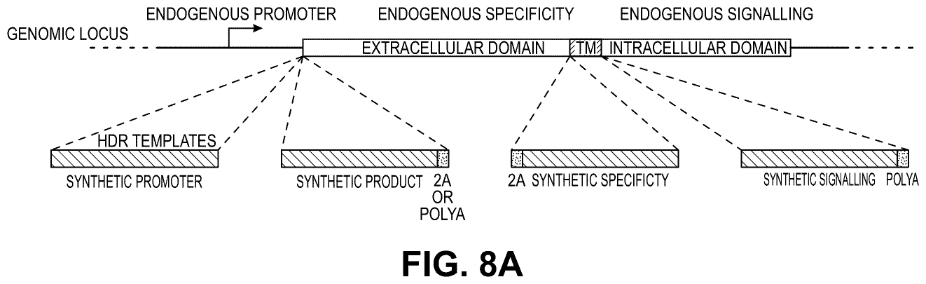

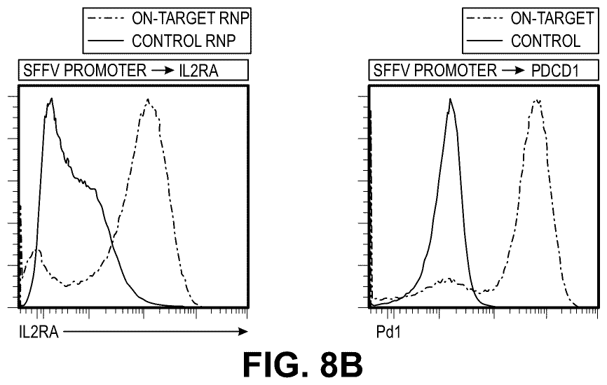

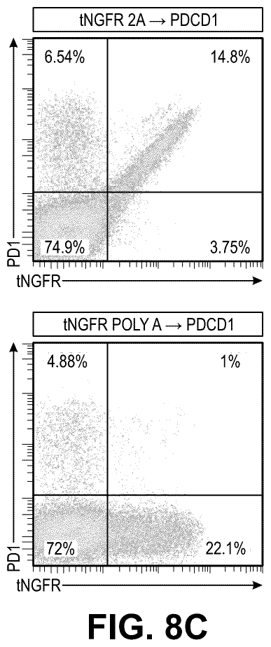

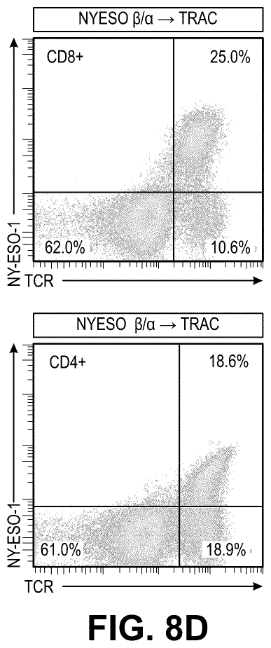

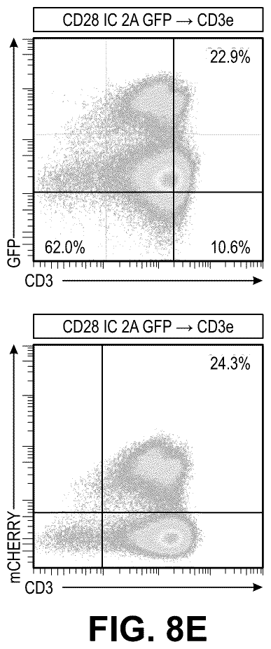

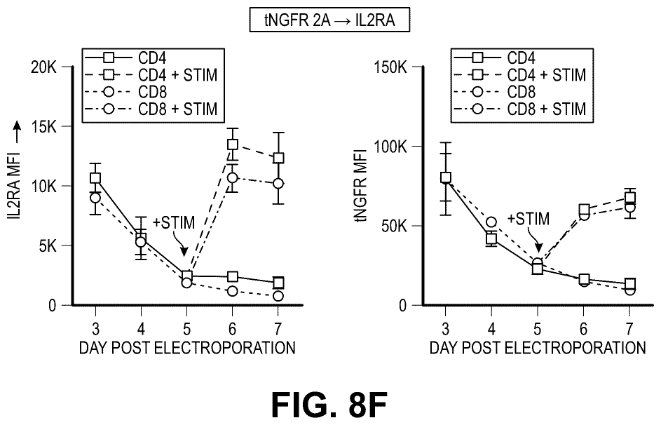

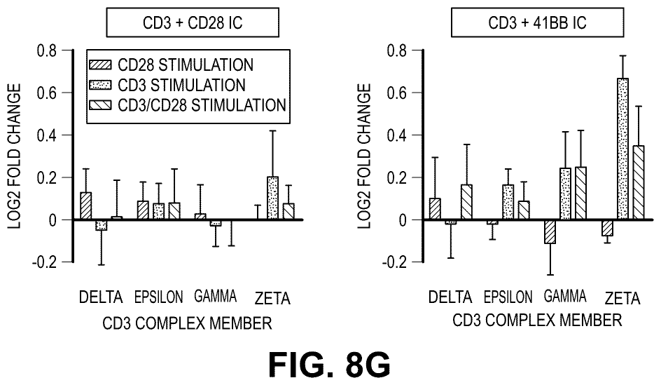

In yet other embodiments, methods are provided for inserting a heterologous coding or non-coding sequence into an endogenous gene locus. Options for these methods include those depicted generically in FIG. 8A. For example, methods for modifying an endogenous cell surface protein gene locus in a human T cell are provided. In some examples, the method comprises (a) introducing into the human T cell (i) a targeted nuclease that cleaves a target region in a nucleic acid sequence in the endogenous cell surface protein gene locus to create an insertion site in the genome of the cell; and (ii) a heterologous nucleic acid sequence comprising a coding or a non-coding sequence, wherein the nucleic acid sequence is flanked by homologous sequences, and (b) allowing homologous recombination to take place, thereby inserting the heterologous nucleic acid sequence in the insertion site to generate a human T cell comprising a modified endogenous cell surface protein gene locus. In some embodiments, the heterologous nucleic acid sequence is inserted in a non-coding sequence of the cell surface protein gene locus.

For example, in some embodiments, a promoter or other transcriptional regulatory non-coding sequence can be inserted by homologous recombination by insertion of a heterologous nucleic acid sequence, for example, an HDR template, to replace an endogenous transcriptional regulatory non-coding sequence in a cell. In some examples, the promoter or other transcriptional regulatory non-coding sequence is inserted into a 5′ noncoding sequence of a cell surface protein locus, such that, upon insertion, the cell surface protein is operatively linked to or under the control of the promoter or other transcriptional regulatory non-coding sequence inserted into the 5′ non-coding sequence of the cell surface receptor locus.

In other embodiments, the HDR template encodes a heterologous protein. Insertion of the coding sequence of the heterologous protein can be achieved along with insertion of a self-cleaving peptide coding sequence positioned to separate the endogenous protein coding sequence from the heterologous protein sequence. Options for insertion are depicted for example in the middle two options of FIG. 8A. In these embodiments, the heterologous coding sequence is inserted just upstream or downstream of the endogenous coding sequence (or an endogenous extracellular domain) with an intervening coding sequence for a self-cleaving peptide. The resulting coding sequence will encode the endogenous polypeptide, self-cleaving peptide and heterologous protein in that order, or alternatively, the heterologous protein, self-cleaving peptide and endogenous polypeptide. This combined protein sequence will be expressed according to the endogenous regulation and will result, following self-cleavage of essentially 1:1 expression of the endogenous polypeptide and the heterologous polypeptide.

In another aspect, one can insert a coding sequence for a heterologous protein followed by a transcriptional terminator sequence (for example including but not limited to a poly A sequence) at or near the amino terminus of the coding sequence for the endogenous polypeptide, resulting in an insertion under the control of the endogenous regulation but as a result of the insertion of the terminator sequence, little or no expression of the endogenous protein. An aspect of this embodiment is depicted for example in the second option of FIG. 8A where Poly A is used.

### Methods of Making Modified Human T Cells

Methods for modifying the genome of a T cell include a method of modifying or editing the genome of a human T cell comprising inserting a nucleic acid sequence or construct encoding a functional domain into a target region of a gene encoding an endogenous cell surface protein in a human T cell. In the methods provided herein, the nucleic acid sequence or construct is inserted into the T cell by introducing into the T cell, (a) a targeted nuclease that cleaves a target region a gene encoding the cell surface protein to create an insertion site in the genome of the T cell; and (b) the nucleic acid sequence encoding a functional domain or a functional fragment thereof, wherein the nucleic acid sequence is incorporated into the insertion site by HDR.

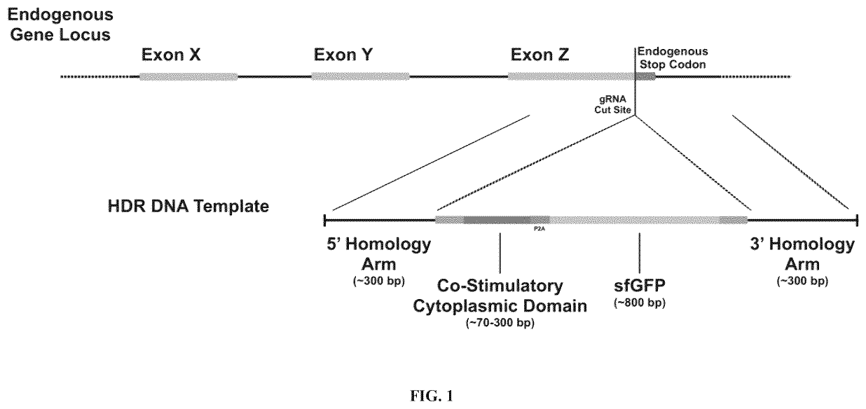

In some embodiments, the target region is in an exon encoding the C-terminus of the endogenous cell surface protein. In some embodiments, for insertion into an exon encoding the C-terminus, the nucleic acid encodes a functional domain, wherein the nucleic acid encoding the functional domain is flanked by homologous sequences. In some embodiments, the construct optionally includes a selectable marker. For example, for insertion into an exon encoding the C-terminus of an endogenous cell surface protein, the nucleic acid construct encodes sequentially, from the N-terminus to the C-terminus, a functional domain or a functional fragment thereof, a self-cleaving peptide and a selectable marker, wherein the nucleic acid construct is flanked by homologous sequences. An exemplary construct is shown in FIG. 1. In some embodiments, one or more endogenous cell surface proteins of a human T cell are modified using any of the methods described herein.

In some embodiments, one or more linker sequences separate the components of the nucleic acid construct. The linker sequence can be two, three, four, five, six, seven, eight, nine, ten amino acids or greater in length.

In some embodiments, the nucleic acid encoding the functional domain further comprises a furin cleavage sequence. Including a furin cleavage sequence, for example, a furin cleavage sequence that precedes a self-cleavage peptide in a protein, facilitates removal of the self-cleavage peptide from the protein.

Upon insertion by homologous recombination, the construct encoding the functional domain or functional fragment thereof, is under the control of the endogenous promoter of the cell surface protein. Once the construct is incorporated into the genome of the T cell by HDR, and under the control of the endogenous promoter of the cell surface protein, the T cells can be cultured under conditions that allow transcription of the inserted construct into a single mRNA sequence encoding a fusion polypeptide. The fusion polypeptide comprises the endogenous cell surface protein and the functional domain or functional fragment thereof, in that order. By inserting the construct into the exon encoding the C-terminus of the endogenous cell surface protein, the remaining exons of the cell surface protein receptor gene are spliced together with the exon encoding the C-terminus into the final mRNA sequence.

In some embodiments where the construct encodes a selectable marker, upon insertion, the construct encoding the functional domain or functional fragment thereof, the self-cleaving peptide and the selectable marker, in that order, is under the control of the endogenous promoter of the cell surface protein. Once the construct is incorporated into the genome of the T cell by HDR, and under the control of the endogenous promoter of the cell surface protein, the T cells can be cultured under conditions that allow transcription of the inserted construct into a single mRNA sequence encoding a fusion polypeptide. The fusion polypeptide comprises the endogenous cell surface protein, the functional domain or functional fragment thereof, the self-cleaving peptide and the selectable marker, in that order. By inserting the construct into the exon encoding the C-terminus of the endogenous cell surface protein, the remaining exons of the cell surface protein receptor gene are spliced together with the exon encoding the C-terminus into the final mRNA sequence. Translation of this mRNA sequence results in expression of one protein that self-cleaves into two, separate polypeptide sequences, i.e., a modified endogenous cell surface protein comprising the full-length endogenous cell surface protein linked to the functional domain or a functional fragment thereof, and a selectable marker. Linking the functional domain to the C-terminus of the endogenous cell surface protein, for example, to the cytoplasmic domain of the endogenous cell surface protein imparts the properties of the functional domain onto the endogenous cell surface protein. Therefore, the full-length modified endogenous cell surface protein retains one or more activities of the endogenous cell surface protein, and has one or more activities associated with the functional domain.

In some embodiments, for insertion into an exon encoding the N-terminus, the nucleic acid encodes a functional domain, wherein the nucleic acid encoding the functional domain is flanked by homologous sequences. In some embodiments, the construct optionally includes a selectable marker. For example, for the nucleic acid construct encodes sequentially, from the N-terminus to the C-terminus, a selectable marker, a self-cleaving peptide and a functional domain or a functional fragment thereof, wherein the nucleic acid construct is flanked by homologous sequences.

In embodiments where the construct encodes a selectable marker, upon insertion by homologous recombination, the construct encoding the selectable marker, the self-cleaving peptide and the functional domain or a functional fragment thereof, in that order, is under the control of the endogenous promoter of the cell surface protein. Once the construct is incorporated into the genome of the T cell by HDR, and under the control of the endogenous promoter of the cell surface protein, the T cells can be cultured under conditions that allow transcription of the inserted construct into a single mRNA sequence encoding a fusion polypeptide. The fusion polypeptide comprises the selectable marker, the self-cleaving peptide the functional domain or functional fragment thereof, and the endogenous cell surface protein, in that order. By inserting the construct into the exon encoding the N-terminus of the endogenous cell surface protein, the remaining exons of the cell surface protein receptor gene are spliced together with the exon encoding the N-terminus into the final mRNA sequence. Translation of this mRNA sequence results in expression of one protein that self-cleaves into two, separate polypeptide sequences, i.e., a selectable marker and a modified endogenous cell surface protein comprising the full-length modified endogenous cell surface protein linked to the functional domain or a functional fragment thereof. The full-length modified endogenous cell surface protein has one or more activities of the endogenous cell surface protein and has one or more activities of the functional domain linked to the N-terminus of the endogenous cell surface protein. In some embodiments, the N-terminus of the endogenous cell surface protein is modified to include a functional domain that alters the binding specificity of the endogenous cell surface protein. In some embodiments, the N-terminus is modified to include a dimerization domain.

In some embodiments, the construct for modification of the N-terminus of an endogenous cell surface protein encodes sequentially, from the N-terminus to the C-terminus, a signal sequence, a functional domain or functional fragment thereof, a self-cleaving peptide and a selectable marker, wherein the nucleic acid construct is flanked by homologous sequences. In other embodiments, the construct for modification of the N-terminus of an endogenous cell surface protein encodes sequentially, from the N-terminus to the C-terminus, a selectable marker, a self-cleaving peptide, a signal sequence and a functional domain or a functional fragment thereof, wherein the nucleic acid construct is flanked by homologous sequences.

In some embodiments, for example, a C-terminally modified endogenous cell surface protein, the modified protein has the binding specificity of the endogenous cell surface protein and one or more activities of the functional domain, for example, signaling activity. In some embodiments, the functional domain is a cytoplasmic domain of an intracellular signaling protein or a functional fragment thereof. Therefore, in some embodiments, a modified endogenous cell surface protein comprising a functional domain linked to the C-terminus of the endogenous cell surface protein has the binding specificity of the endogenous cell surface protein and the intracellular signaling activity of the cytoplasmic domain of an intracellular signaling protein or a functional fragment thereof.

In some embodiments, the endogenous cell surface protein is selected from the group consisting of a T cell receptor (TCR) complex protein, a co-stimulatory receptor, a co-inhibitory receptor, a cytokine receptor and a chemokine receptor.

In some embodiments, the cytoplasmic domain of an intracellular signaling protein can be the cytoplasmic domain from a co-stimulatory receptor or a co-inhibitory receptor. In some embodiments, the endogenous cell surface protein is an inhibitory cell surface protein selected from the group consisting of PD1, CTLA4, LAG3, TIM3, IL10RA, IL10RB, TGFBR1 and TGFBR2, and the cytoplasmic domain is the cytoplasmic domain of an activating protein selected from the group consisting of CD3-Zeta, CD28, IL2RA, IL7RA, IL15RA, IFNGR1, IFNGR2, 41BB, or a functional fragment thereof. For example, the cytoplasmic domains of TGFBR1 and TGFBR2 can be replaced with the cytoplasmic domains of IFNGR1 and IFNGR2, respectively, such that the immunosuppressive signal of transforming growth factor beta (TGFB) is replaced with a stimulatory signal of interferon gamma (IFNG).

In some embodiments, the endogenous TCR complex protein is selected from the group consisting of the TCR-α chain, the TCR-β chain, the CD3δ chain, the CD3ε chain, the CD3γ chain, and the CD3ζ chain of the endogenous TCR complex of the T cell.

In some embodiments, a heterologous functional domain or a functional fragment thereof is attached to the cytoplasmic domain of a TCR complex protein to produce a modified T cell having the binding specificity of the endogenous TCR of the T cell and the signaling activity of the heterologous functional domain or a functional fragment thereof. In some embodiments, an endogenous TCR complex protein of a human T cell is modified. The method comprises (a) introducing into the human T cell a nuclease that cleaves a target region in an exon encoding the C-terminus of an endogenous TCR complex protein in the human T cell to create an insertion site in the genome of the cell; and a heterologous nucleic acid sequence encoding, from the N-terminus to the C-terminus (1) the cytoplasmic domain of a co-stimulatory receptor or a functional fragment thereof; (2) a self-cleaving peptide sequence; and (3) a selectable marker, wherein the nucleic acid sequence is flanked by homologous sequences, and (b) allowing homologous recombination to take place, thereby inserting the nucleic acid sequence in the insertion site to generate a modified human T cell, wherein the modified TCR complex of the T cell comprises the modified TCR complex protein, wherein the heterologous cytoplasmic domain of the co-stimulatory receptor or a functional fragment thereof is linked to the cytoplasmic domain of the TCR complex protein, and wherein the modified TCR complex has the antigen-binding specificity of the endogenous TCR and the signaling activity of the cytoplasmic domain of the co-stimulatory receptor or a functional fragment thereof.

In this embodiment, upon insertion, the construct encoding the cytoplasmic domain of a co-stimulatory receptor or a functional fragment thereof, the self-cleaving peptide and the selectable marker, in that order, is under the control of the endogenous promoter of the TCR complex protein. Once the construct is incorporated into the genome of the T cell by HDR, and under the control of the endogenous promoter of the TCR complex protein, the T cells can be cultured under conditions that allow transcription of the inserted construct into a single mRNA sequence encoding a fusion polypeptide. The fusion polypeptide comprises the TCR complex protein, the cytoplasmic domain of a co-stimulatory receptor or a functional fragment thereof, the self-cleaving peptide and the selectable marker, in that order. By inserting the construct into the exon encoding the C-terminus of the endogenous TCR protein, the remaining exons of the gene encoding the endogenous TCR complex protein are spliced together with the exon encoding the C-terminus into the final mRNA sequence. Translation of this mRNA sequence results in expression of one protein that self-cleaves into two, separate polypeptide sequences, i.e., a modified endogenous TCR complex protein comprising the full-length TCR complex protein linked to the cytoplasmic domain of a co-stimulatory receptor or a functional fragment thereof, and a selectable marker. The modified TCR complex of the T cell comprises the modified TCR complex protein comprising the full-length TCR protein linked to the cytoplasmic domain of a co-stimulatory receptor or a functional fragment thereof. By modifying the C-terminal portion of the endogenous TCR protein, the modified TCR complex retains the antigen-binding specificity of the endogenous TCR and has the signaling activity imparted by the cytoplasmic domain of the co-stimulatory receptor or a functional fragment thereof.

In some embodiments, the TCR complex protein is the endogenous CD3δ chain of the endogenous TCR complex and the nucleic acid encoding the cytoplasmic domain of the co-stimulatory receptor or a functional fragment thereof is inserted in exon 5 encoding the C-terminus of the endogenous CD3δ chain. In some embodiments, the TCR complex protein is the endogenous CD3ε chain of the endogenous TCR complex and the nucleic acid encoding the cytoplasmic domain of the co-stimulatory receptor or a functional fragment thereof is inserted in exon 9 encoding the C-terminus of the endogenous CD3ε chain. In some embodiments, the TCR complex protein is the endogenous CD3γ chain of the endogenous TCR complex and the nucleic acid encoding the cytoplasmic domain of the co-stimulatory receptor or a functional fragment thereof is inserted in exon 6 encoding the C-terminus of the endogenous CD3γ chain. In some embodiments, the TCR complex protein is the endogenous CD3ζ/CD247 chain of the endogenous TCR complex and the nucleic acid encoding the cytoplasmic domain of the co-stimulatory receptor or a functional fragment thereof is inserted in exon 8 encoding the C-terminus of the endogenous CD3ζ chain. In some embodiments, the TCR complex protein is the endogenous TCR-α chain of the endogenous TCR complex and the nucleic acid encoding the cytoplasmic domain of the co-stimulatory receptor or a functional fragment thereof is inserted in exon 8 of the TRAC gene, which encodes the C-terminus of the TCR-α chain. In some embodiments, the TCR complex protein is the endogenous TCR-β chain of the endogenous TCR complex and the nucleic acid encoding the cytoplasmic domain of the co-stimulatory receptor or a functional fragment thereof is inserted in exon 4 or exon 9 of the TRBC1 or TRBC2 gene, respectively.

In some embodiments, the co-stimulatory receptor is CD28, 41BB, DAP10 or MYD88. Examples of amino acid sequences of cytoplasmic domains of co-stimulatory receptors that can be inserted into an endogenous cell surface protein of a T cell using the methods described herein include, but are not limited to the amino acid sequence of the cytoplasmic domain of human CD28 (GSGGTSGRSKRSRLLHSDYMNMTPRRPGPTRKHYQPYAPPRDFAAYRS) (SEQ ID NO: 1); the amino acid sequence of the cytoplasmic domain of human 41BB (GSGGTSGKRGRKKLLYIFKQPFMRPVQTTQEEDGCSCRFPEEEEGGCEL) (SEQ ID NO: 2); the amino acid sequence of the cytoplasmic domain of human MyD88 (GSGGTSGMDFEYLEIRQLETQADPTGRLLDAWQGRPGASVGRLLELLTKLGRDDVL LELGPSIEEDCQKYILKQQQEEAEKPLQVAAVDSSVPRTAELAGITTLDDPLG (SEQ ID NO: 3); and the amino acid sequence of the cytoplasmic domain of human Dap10 (GSGGTSGLCARPRRSPAQEDGKVYINMPGRG) (SEQ ID NO: 4). Any one of SEQ ID Nos: 1-4 can be further comprise a linker and/or a furin cleavage sequence (RAKR) (SEQ ID NO: 5). Examples of such sequences include, but are not limited to SEQ ID NO: 6 which comprises the cytoplasmic domain of human CD28; SEQ ID NO: 7 which comprises the cytoplasmic domain of human 41BB; SEQ ID NO: 8 which comprises the cytoplasmic domain of human MyD88 and SEQ ID NO: 9 which comprises the cytoplasmic domain of human Dap10. It is understood that amino acid sequences consisting of or comprising a functional domain can be linked to or inserted into endogenous cell surface proteins using any of the methods described herein.

Nucleic acid sequences encoding the cytoplasmic domain of a co-stimulatory receptor or a functional fragment thereof are also provided. Exemplary nucleic acids sequences include, but are not limited to, SEQ ID NO: 10 which encodes the cytoplasmic domain of human CD28, SEQ ID NO: 11, the cytoplasmic domain of human 41BB; SEQ ID NO: 12 which encodes the cytoplasmic domain of human MyD88; and SEQ ID NO: 13 which encodes the cytoplasmic domain of human Dap10.

In some embodiments, one or more TCR complex proteins are modified by inserting the nucleic acid sequence into an exon encoding the C-terminus of an endogenous TCR complex protein. In some embodiments, the TCR complex comprises one or more modified endogenous TCR complex proteins, wherein each of the TCR complex proteins is linked to the cytoplasmic domain of a co-stimulatory receptor or a functional fragment thereof. In some embodiments, each of the one or more TCR complex proteins is linked to a unique cytoplasmic domain of a co-stimulatory receptor or functional fragment thereof, In some embodiments, the nucleic acid sequence encoding the cytoplasmic domain of the co-stimulatory receptor or a functional fragment thereof is inserted downstream of the last amino acid of the endogenous TCR complex protein and upstream of the stop codon for the endogenous TCR complex protein.

In some embodiments, depending on the insertion site for the functional domain or a functional fragment thereof, the construct may include a nucleic acid sequence encoding one, two, three, four, five, six, seven, eight, nine, ten or more codons of the C-terminal end, upstream of the stop codon for the endogenous cell surface receptor.

It is understood that the methods described above for modifying one or more endogenous cell surface proteins expressed by T cells can be used to modify one or more endogenous cell surface proteins expressed by other human non-T cells. Although an endogenous cell surface protein in any human cell, for example, a human T cell, can be modified by inserting a nucleic acid encoding a functional domain or a functional fragment thereof directly into the coding sequence of the endogenous cell surface protein, in some embodiments, the entire endogenous cell surface protein can be replaced with a modified full-length endogenous cell surface protein comprising a functional domain. In these embodiments, a nucleic acid construct comprising a heterologous nucleic acid sequence encoding the full-length endogenous protein linked to a nucleic acid encoding the functional domain or a functional fragment thereof can be used.

In some embodiments, the TCR complex of a T cell can be modified by inserting a full-length T cell receptor TCR-β chain linked to the cytoplasmic domain of a co-stimulatory receptor or a functional fragment thereof and a variable region of a TCR-α chain into a target region in exon 1 of a TCR-α subunit constant gene (TRAC). Upon insertion, the modified TCR complex expressed by the T cell has the antigen-specificity of the TCR comprising the T cell receptor TCR-β chain linked to the heterologous cytoplasmic domain of the co-stimulatory receptor or a functional fragment thereof, and the TCR-α chain comprising the variable region of the TCR-α chain introduced into the cell, and the signaling activity of the cytoplasmic domain of the co-stimulatory receptor or a functional fragment thereof C-terminally linked to the cytoplasmic domain of the TCR-β chain.

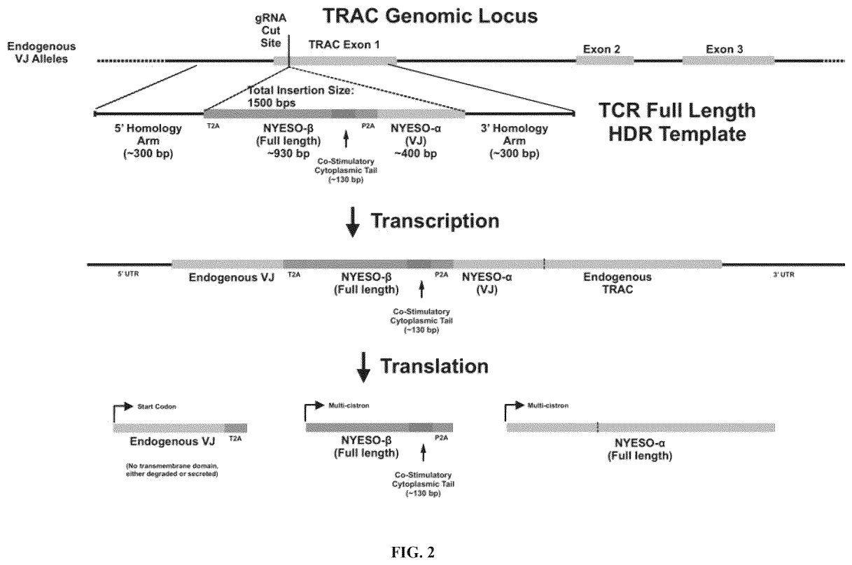

In some embodiments, the method comprises (a) introducing into the human T cell (i) a targeted nuclease that cleaves a target region in exon 1 of a TCR-alpha subunit constant gene (TRAC) in the human T cell to create an insertion site in the genome of the cell; (ii) a heterologous nucleic acid sequence encoding, from the N-terminus to the C-terminus, (1) a first self-cleaving peptide sequence; (2) a full-length T cell receptor (TCR)-β chain; (3) the cytoplasmic domain of a co-stimulatory receptor or a functional fragment thereof; (4) a second self-cleaving peptide sequence; (5) a variable region of a TCR-α chain; and (6) a portion of the N-terminus of the endogenous TCR-α chain, wherein the nucleic acid sequence is flanked by homologous sequences; and (b) allowing recombination to occur, thereby inserting the nucleic acid sequence in the insertion site to generate a modified human T cell, wherein the heterologous cytoplasmic domain of a co-stimulatory receptor or a functional fragment thereof is linked to the cytoplasmic domain of the full-length T cell receptor (TCR)-β chain, wherein the modified TCR of the T cell is antigen-specific and wherein the modified TCR has the signaling activity of the cytoplasmic domain of the co-stimulatory receptor or a functional fragment thereof. An exemplary construct is shown in FIG. 2. In this embodiment, upon insertion, the construct encoding the first self-cleaving peptide, the heterologous full-length TCR-β chain, the cytoplasmic domain of a co-stimulatory receptor or a functional fragment thereof, the second self-cleaving peptide, the heterologous full-length TCR-α chain, and the portion of the N-terminus of the endogenous TCR-α subunit, in that order, is under the control of the endogenous TCR-α promoter and TCR-α regulatory elements. Once the construct is incorporated into the genome of the T cell and under the control of the endogenous TCR-α promoter, the T cells can be cultured under conditions that allow transcription of the inserted construct into a single mRNA sequence encoding a fusion polypeptide. The fusion polypeptide comprises the first self-cleaving peptide, the heterologous full-length TCR-β chain, the cytoplasmic domain of a co-stimulatory receptor or a functional fragment thereof, the second self-cleaving peptide, the heterologous full-length TCR-α chain, and the portion of the N-terminus of the endogenous TCR-α subunit, in that order.

By inserting the construct into exon 1 of the TRAC gene, the remaining exons of the TRAC gene (exons 2 and 3) are spliced together with exon 1 into the final mRNA sequence. Translation of this mRNA sequence results in expression of one protein that self-cleaves into three, separate polypeptide sequences, i.e., an inactive, endogenous variable region peptide lacking a transmembrane domain, (which can be, e.g., degraded in the endoplasmic reticulum or secreted following translation), a full-length antigen-specific TCR-chain linked to the cytoplasmic domain of a co-stimulatory receptor or a functional fragment thereof, and a full length antigen-specific TCR-α chain. The full-length antigen specific TCR-β chain linked to the cytoplasmic domain of a co-stimulatory receptor or a functional fragment thereof and the full length antigen-specific TCR-α chain form a TCR with desired antigen-specificity and the signaling activity imparted by the cytoplasmic domain of a co-stimulatory receptor or a functional fragment thereof.

Depending on the insertion site in the TRAC gene, the size of the nucleic acid encoding the N-terminal portion of the endogenous TCR-α subunit can vary. The size of the nucleic acid encoding the N-terminal portion of the endogenous TCR-α subunit will depend on the number of nucleotides in the endogenous TRAC nucleic acid sequence between the start of TRAC exon 1 and the targeted insertion site.

In some embodiments, the method comprises (a) introducing into the human T cell (i) a targeted nuclease that cleaves a target region in exon 1 of either a TCR-beta subunit constant gene 1 (TRBC1), a TCR beta subunit constant gene 2 (TRBC2), or both in the human T cell to create an insertion site in the genome of the cell; (ii) a heterologous nucleic acid sequence encoding, from the N-terminus to the C-terminus, (1) a first self-cleaving peptide sequence; (2) a full-length T cell receptor (TCR)-α chain; (3) the cytoplasmic domain of a co-stimulatory receptor or a functional fragment thereof; (4) a second self-cleaving peptide sequence; (5) a variable region of a TCR-β chain; and (6) a portion of the N-terminus of the endogenous TCR-β chain, wherein the nucleic acid sequence is flanked by homologous sequences; and (b) allowing recombination to occur, thereby inserting the nucleic acid sequence in the insertion site to generate a modified human T cell, wherein the heterologous cytoplasmic domain of a co-stimulatory receptor or a functional fragment thereof is linked to the cytoplasmic domain of the full-length T cell receptor (TCR)-α chain, wherein the modified TCR of the T cell is antigen-specific and wherein the modified TCR has the signaling activity of the cytoplasmic domain of the co-stimulatory receptor or a functional fragment thereof.

In this embodiment, upon insertion, the construct encoding the first self-cleaving peptide, the heterologous full-length TCR-α chain, the cytoplasmic domain of a co-stimulatory receptor or a functional fragment thereof, the second self-cleaving peptide, the variable region of the TCR-β chain, and the portion of the N-terminus of the endogenous TCR-β subunit constant region 1 or 2, in that order, is under the control of the endogenous TCR-β promoter and TCR-regulatory elements. Once the construct is incorporated into the genome of the T cell and under the control of the endogenous TCR-β promoter, the T cells can be cultured under conditions that allow transcription of the inserted construct into a single mRNA sequence encoding a fusion polypeptide. The fusion polypeptide comprises the first self-cleaving peptide, the heterologous full-length TCR-α chain, the cytoplasmic domain of a co-stimulatory receptor or a functional fragment thereof, the second self-cleaving peptide, the heterologous full-length TCR-β chain, and the portion of the N-terminus of the endogenous TCR-β subunit, in that order.

By inserting the construct into exon 1 of a TRBC gene, for example, TRBC1, or TRBC2, the remaining exons of the TRBC gene are spliced together with exon 1 into the final mRNA sequence. Translation of this mRNA sequence results in expression of one protein that self-cleaves into three, separate polypeptide sequences, i.e., an inactive, endogenous variable region peptide lacking a transmembrane domain, (which can be, e.g., degraded in the endoplasmic reticulum or secreted following translation), a full-length antigen-specific TCR-α chain linked to the cytoplasmic domain of a co-stimulatory receptor or a functional fragment thereof, and a full length antigen-specific TCR-β chain. The full-length antigen specific TCR-α chain linked to the cytoplasmic domain of a co-stimulatory receptor or a functional fragment thereof and the full length antigen-specific TCR-β chain form a TCR with desired antigen-specificity and the signaling activity imparted by the cytoplasmic domain of a co-stimulatory receptor or a functional fragment thereof.

Depending on the insertion site in the TRBC gene, the size of the nucleic acid encoding the N-terminal portion of the endogenous TCR-β subunit can vary. The size of the nucleic acid encoding the N-terminal portion of the endogenous TCR-β subunit will depend on the number of nucleotides in the endogenous TRBC nucleic acid sequence between the start of TRBC exon 1 and the targeted insertion site.

In some embodiments, if the endogenous TCR of the T cell is replaced to maintain the antigen-specificity of the TCR, while acquiring one or more activities of the cytoplasmic domain of a co-stimulatory receptor, the endogenous TCR complex of the T cell can be modified by linking a heterologous cytoplasmic domain of a co-stimulatory receptor or a functional fragment thereof to the cytoplasmic domain of the endogenous TCR-β chain or the cytoplasmic domain of the endogenous TCR-α chain. In some embodiments, the method comprises inserting a nucleic acid encoding an endogenous T cell receptor TCR-β chain linked to the cytoplasmic domain of a co-stimulatory receptor or a functional fragment thereof and a nucleic acid encoding variable region of an endogenous TCR-α chain into a target region in exon 1 of a TCR-α subunit constant gene (TRAC). Upon insertion, the modified TCR complex of the T cell has the antigen-specificity of the TCR comprising the endogenous T cell receptor TCR-β chain and the endogenous TCR-α chain and the signaling activity of the cytoplasmic domain of the co-stimulatory receptor or a functional fragment thereof C-terminally linked to the endogenous T cell receptor TCR-β chain. Therefore, in some embodiments, the endogenous sequence of the TCR of a T cell from a subject, that has antigen specificity for a target antigen in the subject, can be replaced with a modified TCR comprising a modified endogenous TCR-β chain linked to the cytoplasmic domain of a co-stimulatory receptor or a functional fragment thereof and the endogenous TCR-α chain. By maintaining the endogenous sequence of the TCR in the T cell, the modified T cells administered to the subject have enhanced signaling properties while retaining the antigen specificity of the endogenous TCR for the target antigen in the subject. This method can be used to produce modified T cells with enhanced signaling activity without altering the antigen-binding specificity of the T cells.

In embodiments where altering the antigen-binding specificity and the signaling activity of the T cell is desired, the endogenous TCR in the T cell is replaced with a heterologous TCR that recognizes a specific antigen that is not recognized by the endogenous TCR, for example, a cancer-specific antigen in the subject. Therefore, the TCR complex of a T cell can be modified by inserting a heterologous T cell receptor TCR-β chain linked to the cytoplasmic domain of a co-stimulatory receptor or a functional fragment thereof and a variable region of a heterologous TCR-α chain into a target region in exon 1 of a TCR-α subunit constant gene (TRAC). Upon insertion, the modified TCR complex of the T cell has the antigen-specificity of the TCR comprising the heterologous T cell receptor TCR-β chain and the TCR-α chain comprising the variable region of the heterologous TCR-α chain and the signaling activity of the cytoplasmic domain of the co-stimulatory receptor or a functional fragment thereof C-terminally linked to the heterologous T cell receptor TCR-β chain. In other embodiments, the TCR complex of a T cell can be modified by inserting a heterologous T cell receptor TCR-α chain linked to the cytoplasmic domain of a co-stimulatory receptor or a functional fragment thereof and a variable region of a heterologous TCR-β chain into a target region in exon 1 of a TCR-β subunit constant gene. Upon insertion, the modified TCR complex of the T cell has the antigen-specificity of the TCR comprising the heterologous T cell receptor TCR-β chain and the TCR-α chain comprising the variable region of the heterologous TCR-α chain and the signaling activity of the cytoplasmic domain of the co-stimulatory receptor or a functional fragment thereof C-terminally linked to the heterologous T cell receptor TCR-α chain. This method can be used to produce modified T cells with enhanced signaling activity and antigen-binding specificity for a target antigen.

Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (See, for example, Chng et al. “Cleavage efficient 2A peptides for high level monoclonal antibody expression in CHO cells,” MAbs 7(2): 403-412 (2015)). In some embodiments, the first and second self-cleaving peptides are the same. In other embodiments, the first and second self-cleaving peptides are different.

In the methods provided herein, the full-length TCR-chain comprises variable (V), diversity (D) and joining (J) alleles. In the methods provided herein, the variable region of a heterologous TCR-α chain comprises V and J alleles. See, for example, Kuby, J., Immunology, 7^{th} Ed., W.H. Freeman & Co., New York (2013).

In some embodiments, each of the 5′ and the 3′ ends of the nucleic acid sequence comprise nucleotide sequences that are homologous to genomic sequences flanking a target region in the genome of a T cell, for example, a target region in a gene encoding an endogenous cell surface protein. In some embodiments, the target region is in an exon encoding the C-terminus of the endogenous cell surface protein. In some embodiments, the target region is in an exon encoding the N-terminus of the endogenous cell surface protein. In some examples, the target region is in exon 1 of the TRAC gene. In other examples, the target region is in exon 1 of TRBC1 or exon 1 of TRBC2. In some cases, a nucleotide sequence that is homologous to a genomic sequence is about 50 to 1000 nucleotides in length. In some cases, a nucleotide sequence that is homologous to a genomic sequence, or a portion thereof, is at least 80%, 90%, 95%, complementary to the genomic sequence. In some embodiments, the 5′ and 3′ ends of the nucleic acid sequence comprise nucleotide sequences that are homologous to genomic sequences at an insertion site in an exon encoding the C-terminus of the endogenous cell surface protein or in an exon encoding the N-terminus of the endogenous cell surface protein. In some embodiments, the insertion site is in exon 1 of the TRAC gene.

In some cases, the nucleic acid sequence is introduced into the cell as a linear DNA template. In some cases, the nucleic acid sequence is introduced into the cell as a double-stranded DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA. In some cases, the DNA template is a double-stranded or single-stranded plasmid or mini-circle. In some cases, the nucleic acid is introduced into the cell as a plasmid, a mini-plasmidor in an adeno-associated viral (AAV) vector. In some embodiments, the targeted nuclease is selected from the group consisting of an RNA-guided nuclease domain, a transcription activator-like effector nuclease (TALEN), a zinc finger nuclease (ZFN) and a megaTAL. In some embodiments, the targeted nuclease is an RNA-guided nuclease domain. In some embodiments, the RNA-guided nuclease is a Cas9 nuclease and the method further comprises introducing into the cell a guide RNA that specifically hybridizes to a target region in the genome of the T cell. In other embodiments, the RNA-guided nuclease is a Cas9 nuclease and the method further comprises introducing into the cell a guide RNA that specifically hybridizes to a target region in the genome of the T cell.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence.

Generally, the DNA targeting sequence is designed to complement (e.g., perfectly complement) or substantially complement the target DNA sequence. In some cases, the DNA targeting sequence can incorporate wobble or degenerate bases to bind multiple genetic elements. In some cases, the 19 nucleotides at the 3′ or 5′ end of the binding region are perfectly complementary to the target genetic element or elements. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%).

In some embodiments, the Cas9 protein can be in an active endonuclease form, such that when bound to target nucleic acid as part of a complex with a guide RNA or part of a complex with a DNA template, a double strand break is introduced into the target nucleic acid. In the methods provided herein, a Cas9 polypeptide or a nucleic acid encoding a Cas9 polypeptide can be introduced into the T cell. The double strand break can be repaired by HDR to insert the DNA template into the genome of the T cell. Various Cas9 nucleases can be utilized in the methods described herein. For example, a Cas9 nuclease that requires an NGG protospacer adjacent motif (PAM) immediately 3′ of the region targeted by the guide RNA can be utilized. For example, such Cas9 nucleases can be targeted to a region encoding the N-terminus or the C-terminus of an endogenous cell surface protein that contains an NGG sequence. Such nucleases can also be targeted to exon 1 of the TRAC or exon 1 of the TRBC that contains an NGG sequence. As another example, Cas9 proteins with orthogonal PAM motif requirements can be used to target sequences that do not have an adjacent NGG PAM sequence. Exemplary Cas9 proteins with orthogonal PAM sequence specificities include, but are not limited to those described in Esvelt et al., Nature Methods 10:1116-1121 (2013).

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation (See, for example, Ran et al. “Double nicking by RNA-guided CRISPR Cas9 for enhanced genome editing specificity,” Cell 154(6): 1380-1389 (2013)).

In some embodiments, the targeted nuclease (for example, Cas9), the guide RNA and the nucleic acid sequence are introduced into the cell as a ribonucleoprotein complex (RNP)-DNA template complex, wherein the RNP-DNA template complex comprises: (i) the RNP, wherein the RNP comprises the targeted nuclease and the guide RNA; and (ii) the nucleic acid sequence.

In some embodiments, the molar ratio of RNP to DNA template can be from about 3:1 to about 100:1. For example, the molar ratio can be from about 5:1 to 10:1, from about 5:1 to about 15:1, 5:1 to about 20:1; 5:1 to about 25:1; from about 8:1 to about 12:1; from about 8:1 to about 15:1, from about 8:1 to about 20:1, or from about 8:1 to about 25:1.

In some embodiments, the DNA template in the RNP-DNA template complex is at a concentration of about 2.5 pM to about 25 pM. In some embodiments, the amount of DNA template is about 1 μg to about 10 μg.

In some cases, the RNP-DNA template complex is formed by incubating the RNP with the DNA template for less than about one minute to about thirty minutes, at a temperature of about 20° C. to about 25° C. In some embodiments, the RNP-DNA template complex and the cell are mixed prior to introducing the RNP-DNA template complex into the cell.

In some embodiments the nucleic acid sequence or the RNP-DNA template complex is introduced into the T cells by electroporation. Methods, compositions, and devices for electroporating cells to introduce a RNP-DNA template complex can include those described in the examples herein. Additional or alternative methods, compositions, and devices for electroporating cells to introduce a RNP-DNA template complex can include those described in WO/2006/001614 or Kim, J. A. et al. Biosens. Bioelectron. 23, 1353-1360 (2008). Additional or alternative methods, compositions, and devices for electroporating cells to introduce a RNP-DNA template complex can include those described in U.S. Patent Appl. Pub. Nos. 2006/0094095; 2005/0064596; or 2006/0087522. Additional or alternative methods, compositions, and devices for electroporating cells to introduce a RNP-DNA template complex can include those described in Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842. Additional or alternative methods, compositions, and devices for electroporating cells to introduce a RNP-DNA template complex can include those described in Geng, T. et al., J. Control Release 144, 91-100 (2010); and Wang, J., et al. Lab. Chip 10, 2057-2061 (2010).

In some embodiments, the nucleic acid sequence or RNP-DNA template complex are introduced into about 1×10⁵ to about 2×10⁶ cells T cells. For example, the nucleic acid sequence or RNP-DNA template complex can be introduced into about 1×10⁵ cells to about 5×10⁵ cells, about 1×10⁵ cells to about 1×10⁶ cells, 1×10⁵ cells to about 1.5×10⁶ cells, 1×10⁵ cells to about 2×10⁶ cells, about 1×10⁶ cells to about 1.5×10⁶ cells or about 1×10⁶ cells to about 2×10⁶ cells.

In some embodiments, the RNP is delivered to the cells in the presence of an anionic polymer. In some embodiments, the anionic polymer is an anionic polypeptide or an anionic polysaccharide. In some embodiments, the anionic polymer is an anionic polypeptide (e.g., a polyglutamic acid (PGA), a polyaspartic acid, or polycarboxyglutamic acid). In some embodiments, the anionic polymer is an anionic polysaccharide (e.g., hyaluronic acid (HA), heparin, heparin sulfate, or glycosaminoglycan). In some embodiments, the anionic polymer is poly(acrylic acid) (PAA), poly(methacrylic acid) (PMAA), poly(styrene sulfonate), or polyphosphate. In some embodiments, the anionic polymer has a molecular weight of at least 15 kDa (e.g., between 15 kDa and 50 kDa). In some embodiments, the anionic polymer and the Cas protein are in a molar ratio of between 10:1 and 120:1, respectively (e.g., 10:1, 20:1, 30:1, 40:1, 50:1, 60:1, 70:1, 80:1, 90:1, 100:1, 110:1, or, 120:1). In some embodiments of this aspect, the molar ratio of sgRNA:Cas protein is between 0.25:1 and 4:1 (e.g., 0.25:1, 0.5:1, 1:1, 1.2:1, 1.4:1, 1.6:1, 1.8:1, 2:1, 2.2:1, 2.4:1, 2.6:1, 2.8:1, 3:1, 3.2:1, 3.4:1, 3.6:1, 3.8:1, or 4:1).

In some embodiments, the donor template comprising a homology directed repair (HDR) template and one or more DNA-binding protein target sequences. In some embodiments, the donor template has a “shuttle sequence” i.e., one DNA-binding protein target sequence and one or more protospacer adjacent motif (PAM). The complex containing the DNA-binding protein (e.g., a RNA-guided nuclease), the donor gRNA, and the donor template can “shuttle” the donor template, without cleavage of the DNA-binding protein target sequence, to the desired intracellular location (e.g., the nucleus) such that the HDR template can integrate into the cleaved target nucleic acid. In some embodiments, the DNA-binding protein target sequence and the PAM are located at the 5′ terminus of the HDR template. Particularly, in some embodiments, the PAM can be located at the 5′ terminus of the DNA-binding protein target sequence. In other embodiments, the PAM can be located at the 3′ terminus of the DNA-binding protein target sequence. In some embodiments, the DNA-binding protein target sequence and the PAM are located at the 3′ terminus of the HDR template. Particularly, in some embodiments, the PAM can be located at the 5′ terminus of the DNA-binding protein target sequence. In other embodiments, the PAM is located at the 3′ terminus of the DNA-binding protein target sequence. In some embodiments, the donor template has two DNA-binding protein target sequences and two PAMs. Particularly, in some embodiments, a first DNA-binding protein target sequence and a first PAM are located at the 5′ terminus of the HDR template and a second DNA-binding protein target sequence and a second PAM are located at the 3′ terminus of the HDR template. In some embodiments, the first PAM is located at the 5′ terminus of the first DNA-binding protein target sequence and the second PAM is located at the 5′ of the second DNA-binding protein target sequence. In other embodiments, the first PAM is located at the 5′ terminus of the first DNA-binding protein target sequence and the second PAM is located at the 3′ of the second DNA-binding protein target sequence. In yet other embodiments, the first PAM is located at the 3′ terminus of the first DNA-binding protein target sequence and the second PAM is located at the 5′ of the second DNA-binding protein target sequence. In yet other embodiments, the first PAM is located at the 3′ terminus of the first DNA-binding protein target sequence and the second PAM is located at the 3′ of the second DNA-binding protein target sequence.

In some embodiments, the human cell is a hematopoietic cell, for example, an immune cell, such as a hematopoietic stem cells, a T cell, a B cell, a macrophage, a natural killer (NK) cell or dendritic cell.

In the methods and compositions provided herein, the human T cells can be primary T cells. In some embodiments, the T cell is a regulatory T cell, an effector T cell, or a naïve T cell. In some embodiments, the effector T cell is a CD8⁺ T cell. In some embodiments, the T cell is an CD4+ cell. In some embodiments, the T cell is a CD4⁺CD8⁺ T cell. In some embodiments, the T cell is a CD4⁻CD8⁻ T cell. In some embodiments, the T cell is a T cell that expresses a TCR receptor or differentiates into a T cell that expresses a TCR receptor. Populations of any of the cells modified by any of the methods described herein are also provided. The cell can be in vitro, ex vivo or in vivo. In some cases, T cells are removed from a subject, modified using any of the methods described herein and administered to the patient. In other cases, any of the constructs described herein is delivered to the patient in vivo, for example, via nanoparticle delivery. See, for example, U.S. Pat. No. 9,737,604; Zhang et al. “Lipid nanoparticle-mediated efficient delivery of CRISPR/Cas9 for tumor therapy,” NPG Asia Materials Volume 9, page e441 (2017); and Miller “Nanoparticles improve economic mileage for CARS,” Science Translational Medicine 9(387), eaan2784 DOI: 10.1126/scitranslmed.aan2784. In some embodiments, the constructs can be targeted to tumors or endogenous immune cells subsets in the circulation that can migrate actively into tumors, for example, via in vivo targeted nanoparticle delivery. See, for example, Schmid et al. “T cell targeting nanoparticles focus delivery of immunotherapy to improve antitumor immunity,” Nature Communications 8, Article Number: 1747 (2017) doi: 10.1038/s41467-017-01830-8.

In some embodiments, the modified T cells are cultured under conditions that allow expression of the modified endogenous cell surface protein. In other embodiments, the T cells are cultured under conditions effective for expanding the population of modified cells. In some embodiments, T cells that express the antigen-specific T cell receptor are purified.

### Compositions

Also provided are human T cells comprising a heterologous polypeptide, a heterologous functional domain or a functional fragment thereof. For example, provided herein is a modified T cell comprising a heterologous nucleic acid encoding a functional domain or a functional fragment thereof integrated into an exon encoding the C-terminus of an endogenous cell surface receptor or an exon encoding the N-terminus of an endogenous cell surface receptor. For example, provided herein is a modified T cell comprising a heterologous nucleic acid sequence encoding, from the N-terminus to the C-terminus, (i) a functional domain or a functional fragment thereof; (ii) a self-cleaving peptide sequence; and (iii) a selectable marker, wherein the nucleic acid sequence is integrated into an exon encoding the C-terminus of an endogenous cell surface receptor. Also provided is a modified T cell comprising a heterologous nucleic acid sequence encoding, from the N-terminus to the C-terminus, (i) a selectable marker; (ii) a self-cleaving peptide sequence; and (iii) a functional domain or a functional fragment thereof, wherein the nucleic acid sequence is integrated into an exon encoding the N-terminus of a nucleic acid encoding an endogenous cell surface receptor.

Also provided is a modified T cell comprising a heterologous nucleic acid sequence encoding, from the N-terminus to the C-terminus, (i) a cytoplasmic domain of an intracellular signaling protein or a functional fragment thereof; (ii) a self-cleaving peptide sequence; and (iii) a selectable marker, wherein the nucleic acid sequence is integrated into an exon encoding the C-terminus of a nucleic acid encoding a TCR complex protein. In some embodiments, the cytoplasmic domain of an intracellular signaling protein is a cytoplasmic domain of a co-stimulatory protein or a cytoplasmic domain of a co-inhibitory protein.

Also provided is a modified T cell comprising a heterologous nucleic acid sequence encoding, from the N-terminus to the C-terminus (1) a first self-cleaving peptide sequence; (2) a full-length T cell receptor (TCR)-β chain; (3) the cytoplasmic domain of a co-stimulatory receptor or a functional fragment thereof; (4) a second self-cleaving peptide sequence; (5) a variable region of a TCR-α chain; and (6) a portion of the N-terminus of the endogenous TCR-α chain, wherein the nucleic acid sequence is integrated into exon 1 of a TCR-alpha subunit constant gene (TRAC).

Also provided is a modified T cell comprising a heterologous nucleic acid sequence encoding, from the N-terminus to the C-terminus (1) a first self-cleaving peptide sequence; (2) a full-length T cell receptor (TCR)-α chain; (3) the cytoplasmic domain of a co-stimulatory receptor or a functional fragment thereof; (4) a second self-cleaving peptide sequence; (5) a variable region of a TCR-β chain; and (6) a portion of the N-terminus of the endogenous TCR-β chain, wherein the nucleic acid sequence is integrated into exon 1 of a TCR-beta subunit constant gene (TRBC).

Any of the human T cells described herein can be produced by any of the methods provided herein. Populations of human T cells produced by any of the methods provided herein are also provided. Further provided is a plurality of human T cells, wherein the genome of at least 1%, 5%, 10%, 20%, 30%, 40%, 50%, 60%, 70%, 80%, 90%, 95%, 99% or greater of the cells comprises a targeted insertion of a heterologous nucleic acid encoding a functional domain, wherein the nucleic acid is inserted into a target region in a nucleic acid encoding an endogenous cell surface receptor. In some embodiments, at least 20%, 30%, 40%, 50%, 60%, 70%, 80%, 90%, 95%, 99% or greater of the cells comprise a heterologous functional domain attached to the cytoplasmic domain of an endogenous cell surface receptor. In some embodiments, the T cells are regulatory T cells, effector T cells, or naïve T cells. In some embodiments, the effector T cells are CD8⁺ T cells. In some embodiments, the effector T cells are CD4⁺CD8⁺ T cells.

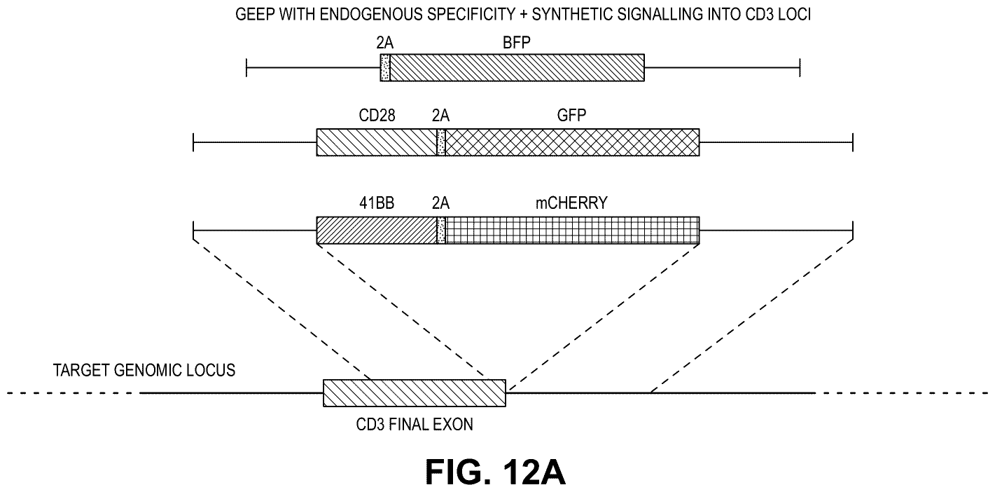

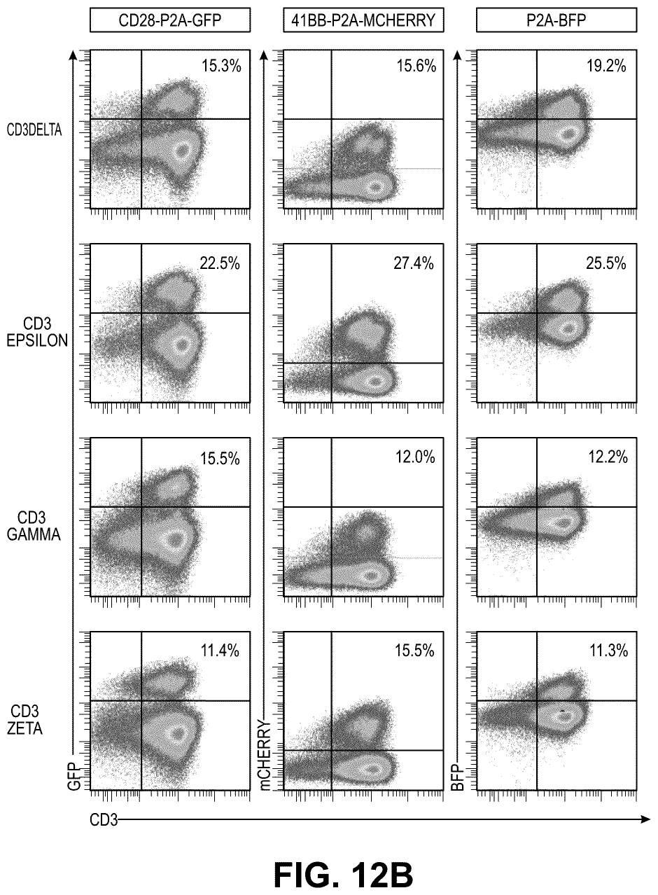

As described in Example 2, a T-cell in which the endogenous CD3 ζ locus was modified such that the encoded CD3ζ protein was fused at its terminus with the 4-1BB intracellular domain (see, e.g., FIG. 12) demonstrated increased proliferation in response to CD3 stimulation in the absence of CD28 costimulation. Accordingly, a human T-cell comprising a heterologous 4-1BB intracellular domain coding sequence operably linked to the CD3ζ protein coding sequence in an endogenous CD3 locus is provided. This results in a CD3 ζ-4-1BB intracellular domain fusion protein encoded in the endogenous CD3 locus. The precise location of the fusion between the CD3ζ protein and the 4-1BB intracellular domain can vary. For example, in some embodiments, the last amino acid of the CD3ζ protein in the fusion can be the last native CD3ζ protein amino acid of the fusion or can be upstream of the last native amino acid such that the CD3ζ protein is truncated at the c-terminus, e.g., for example 1-20 amino acids of the CD3ζ protein are omitted. Similarly, the first amino acid of the fused 4-1BB intracellular domain can vary. For example, the first amino acid of the 4-1BB intracellular domain can be any of the first 5, 10, 15, or 20 amino acids of the following sequence, which is the human 4-1BB intracellular domain: KRGRKKLLYIFKQPFMRPVQTTQEEDGCCRFPEEEEGGCEL (SEQ ID NO:15). In some embodiments, the CD3ζ protein and the 4-1BB intracellular domain are linked via a linking amino acid sequence. An exemplary linking amino acid sequence is RAKRSGSG (SEQ ID NO:16). In some embodiments, the resulting fusion protein comprises all of the above sequence. For example, an exemplary fusion protein is at least 90%, 95%, 99% or 100% identical to

(SEQ ID NO: 17)
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPRGSGGTSGKRGRKKLLYIFKQPFMRPVQTTQEE
DGCSCRFPEEEEGGCELRAKRSGSGATNFSLLKQAGDVEENPGPMVSKG
EEDNMAIIKEFMRFKVHMEGSVNGHEFEIEGEGEGRPYEGTQTAKLKVT
KGGPLPFAWDILSPQFMYGSKAYVKHPADIPDYLKLSFPEGFKWERVMN
FEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVN
IKLDITSHNEDYTIVEQYERAEGRHSTGGMDELYKGTGAGSG.

The addition of a 4-1BB intracellular domain at the C-terminus of CD3zeta provides a scaffold for signaling proteins (i.e. TRAF 1, 2, 3) downstream of 4-1BB to bind and function. By engineering the CD3zeta subunit of the endogenous TCR complex to provide 4-1BB co-stimulation, we preserve all of the advantages of endogenous TCR protein and gene regulation, leverage endogenous polyclonal TCR repertoires (by only modifying the CD3 subunit and leaving the TCR beta and alpha chain untouched), and more precisely limit the effects of 4-1BB co-stimulation to engineered T cell subsets with known specificities. With this modification, we can generate T-cell therapies with more potent and durable antitumor responses.

### Methods of Treatment

Any of the methods and compositions described herein can be used to modify T cells obtained from a human subject. Any of the methods and compositions described herein can be used to modify T cells obtained from a human subject treat or prevent a disease (e.g., cancer, an infectious disease, an autoimmune disease, transplantation rejection, graft vs. host disease or other inflammatory disorder in a subject).

Provided herein is a method of treating cancer in a human subject comprising: a) obtaining T cells from the subject; b) modifying the T cells using any of the methods provided herein to express an antigen-specific TCR complex that recognizes a target antigen in the subject; and c) administering the modified T cells to the subject, wherein the human subject has cancer and the target antigen is a cancer-specific antigen. As used throughout, the phrase “cancer-specific antigen” means an antigen that is unique to cancer cells or is expressed more abundantly in cancer cells than in in non-cancerous cells. In some embodiments, the cancer-specific antigen is a tumor-specific antigen. In some embodiments, the T-cell expresses the above-described CD3 ζ-4-1BB intracellular domain fusion protein from an endogenous CD3 locus. Such T-cells can be administered to a human in need thereof, including for example a human having cancer, in a therapeutically-effective amount. The T-cell can in some embodiments include a heterologous TCR variable region, thereby targeting the T-cell to a cancer epitope in the human. The T-cell can be autologous (purified, modified ex vivo and returned to the human) or autologous, optionally selected to match HLA loci with the human recipient.

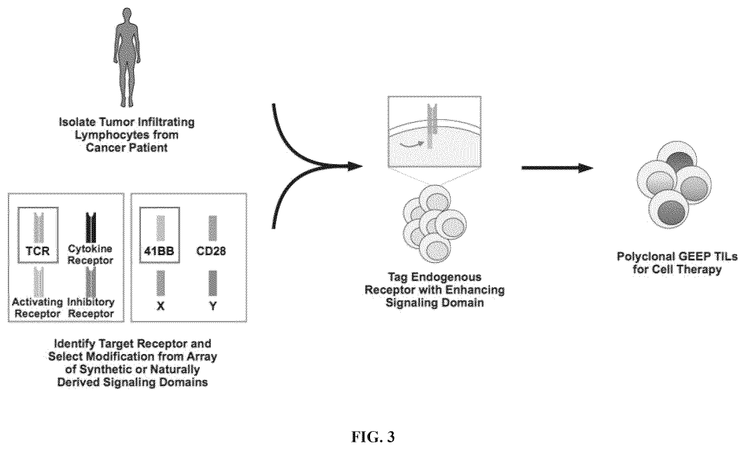

In some embodiments, tumor infiltrating lymphocytes, a heterogeneous and cancer-specific T-cell population, are obtained from a cancer subject and expanded ex vivo. The characteristics of the patient's cancer determine a set of tailored cellular modifications, and these modifications are applied to the tumor infiltrating lymphocytes using any of the methods described herein (See FIG. 3). Modified tumor infiltrating lymphocytes are then reintroduced to the subject. The resulting cell therapy is advantageous over existing cell therapies, namely CAR T-Cells because the methods provided herein non-virally target genetic modifications to the endogenous loci of the protein of interest, as opposed to virally integrating new genetic information randomly throughout the genome. Also, since endogenous TCRs are modified, the TCRs maintain their existing specificity of cancer antigens. Additionally, this strategy also capitalizes on and enhances the function of the patient's natural repertoire of cancer specific T cells, providing a diverse arsenal to eliminate mutagenic cancer cells quickly. Similar strategies are applicable for the treatment of autoimmune and infectious disease.

Also provided herein is a method of treating an autoimmune disease in a human subject comprising: a) obtaining T cells from the subject; b) modifying the T cells using any of the methods provided herein to express a modified antigen-specific TCR complex that recognizes a target antigen in the subject; and c) administering the modified T cells to the subject, wherein the human subject has an autoimmune disorder and the target antigen is antigen associated with the autoimmune disorder. In some embodiments, for example, in a method for treating an autoimmune disorder, the T cells are regulatory T cells or otherwise suppressive T cells.

Also provided herein is a method of treating an infection in a human subject comprising: a) obtaining T cells from the subject; b) modifying the T cells using any of the methods provided herein to express a modified antigen-specific TCR complex that recognizes a target antigen in the subject; and c) administering the modified T cells to the subject, wherein the subject has in infection and the target antigen is an antigen associated with the infection in the subject.

Any of the methods of treatment provided herein can further comprise expanding the population of T cells before the endogenous TCR is replaced with a heterologous TCR. Any of the methods of treatment provided herein can further comprise expanding the population of T cells after the endogenous TCR is replaced with a heterologous TCR and prior to administration to the subject.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

The following examples are provided by way of illustration only and not by way of limitation. Those of skill in the art will readily recognize a variety of non-critical parameters that could be changed or modified to yield essentially the same or similar results.

### Example 1

### Isolation of Human Primary T Cells for Gene Targeting

Primary human T cells were isolated from healthy human donors either from fresh whole blood samples, residuals from leukoreduction chambers after Trima Apheresis (Blood Centers of the Pacific), or leukapheresis products (StemCell). Peripheral blood mononuclear cells (PBMCs) were isolated from whole blood samples by Ficoll centrifugation using SepMate tubes (STEMCELL, per manufacturer's instructions). T cells were isolated from PBMCs from all cell sources by magnetic negative selection using an EasySep Human T Cell Isolation Kit (STEMCELL, per manufacturer's instructions). Unless otherwise noted, isolated T cells were stimulated and used directly (fresh). When frozen cells were used, previously isolated T cells that had been frozen in Bambanker freezing medium (Bulldog Bio) per manufacturer's instructions were thawed, cultured in media without stimulation for 1 day, and then stimulated and handled as described for freshly isolated samples. Fresh healthy human blood donors were consented under protocol approved by the UCSF Committee on Human Research (CHR). Patient samples for gene editing were obtained under a protocol approved by the Yale Internal Review Board (IRB).

### Primary T Cell Culture

Unless otherwise noted, bulk T cells were cultured in XVivo™ 15 medium (STEMCELL) with 5% Fetal Bovine Serum, 50 mM 2-mercaptoethanol, and 10 mM N-Acetyl L-Cystine. Serum free media (ImmunoCult XF T cell expansion media, STEMCELL) without additives, as well as RPMI+10% FBS were used in indicated experiments (FIG. 15). Immediately following isolation, T cells were stimulated for 2 days with anti-human CD3/CD28 magnetic dynabeads (ThermoFisher) at a beads to cells concentration of 1:1, along with a cytokine cocktail of IL-2 at 200 U/mL (UCSF Pharmacy), IL-7 at 5 ng/mL (ThermoFisher), and IL-15 at 5 ng/ml (Life Tech). Following electroporation, T cells were cultured in media with IL-2 at 500 U/mL. Throughout culture T cells were maintained at an approximate density of 1 million cells per mL of media. Every 2-3 days post-electroporation additional media was added, along with additional fresh IL-2 to bring the final concentration to 500 U/mL, and cells were transferred to larger culture vessels as necessary to maintain a density of 1 million cells/mL.

### RNP Production

RNPs were produced by annealing of a two-component gRNA to Cas9, as previously described (Schumann et al. PNAS 112:10437-10442 (2015); and Hultquist et al. Cell Rep. 17:1438-1452 (2016))). Briefly, crRNAs and tracrRNAs were chemically synthesized (Dharmacon, IDT), and recombinant Cas9-NLS, D10A-NLS, or dCas9-NLS were recombinantly produced and purified (QB3 Macrolab). Lyophilized RNA was resuspended in Tris-HCL (7.4 pH) with 150 mM KCl at a concentration of 160 uM, and stored in aliquots at −80 C. crRNA and tracrRNA aliquots were thawed, mixed 1:1 by volume, and incubated at 37 C for 30 min to form an 80 uM gRNA solution. Recombinant Cas9 and variants, stored at 40 uM in 20 mM HEPES-KOH PH 7.5, 150 mM KCl, 10% glycerol, 1 mM DTT, were then mixed 1:1 by volume with the 80 uM gRNA (2:1 gRNA to Cas9 molar ratio) at 37° C. for 15 min to form an RNP at 20 uM. RNPs were generally electroporated immediately after complexing.

dsDNA Homology-Directed Recombination Template (HDRT) Production

Double stranded DNA HDRT sequences were generated from PCR products. Novel HDR sequences were constructed using Gibson Assemblies to place the HDR template sequence, consisting of the homology arms (commonly synthesized as gBlocks from IDT) and the desired insert (such as GFP) into a cloning vector for sequence confirmation and future propagation. These plasmids were used as templates for high-output PCR amplification (Kapa Hotstart polymerase). PCR amplicons (the dsDNA HDRT) were SPRI purified (1.0×) and eluted into a final volume of 3 μL H2O per 100 μL of PCR reaction input. Concentrations of HDRTs were analyzed by nanodrop with a 1:20 dilution. The size of the amplified HDRT was confirmed by gel electrophoresis in a 1.0% agarose gel.

### SSDNA HDRT Production by Exonuclease Digestion

To produce long ssDNA as HDR donors, the DNA of interest was amplified via PCR using one regular, non-modified PCR primer and a second phosphorylated PCR primer. The DNA strand that will be amplified using the phosphorylated primer, will be the strand that will be degraded using this method. This allows to either prepare a single stranded sense or single stranded antisense DNA using the respective phosphorylated PCR primer. To produce the ssDNA strand of interest, the phosphorylated strand of the PCR product was degraded via subsequent treatment with two enzymes, Strandase Mix A and Strandase Mix B, for 5 minutes (per 1 kb) at 37° C., respectively. Enzymes were deactivated by a 5 minute incubation at 80 C. Resulting ssDNA HDR templates were SPRI purified (1.0×) and eluted in H2O. A more detailed protocol for the Guide-It™ Long ssDNA Production System (Takara Bio USA, Inc. #632644) can be found at the manufacturer's website.

### SSDNA HDRT Production by Reverse Synthesis

ssDNA donors were synthesized by reverse transcription of an RNA intermediate followed by hydrolysis of the RNA strand in the resulting RNA: DNA hybrid product, as described in Leonetti et al. http://www.biorxiv.org/content/early/2017/08/21/178905). Briefly, the desired HDR donor was first cloned downstream of a T7 promoter and the T7-HDR donor sequence amplified by PCR. RNA was synthesized by in vitro transcription using HiScribe T7 RNA polymerase (New England Biolabs) and reverse-transcribed using TGIRT-III (InGex). Following reverse transcription, NaOH and EDTA were added to 0.2 M and 0.1 M respectively and RNA hydrolysis carried out at 95° C. for 10 min. The reaction was quenched with HCl, the final ssDNA product purified using Ampure XP magnetic beads (Beckman Coulter) and eluted in sterile RNAse-free H₂O. ssDNA quality was analyzed by capillary electrophoresis (Bioanalyzer, Agilent).

### Primary T Cell Electroporations

RNPs and HDR templates were electroporated 2 days following initial T cell stimulation. T cells were harvested from their culture vessels and magnetic CD3/CD28 dynabeads were removed by placing cells on a magnet for 2 minutes. Immediately prior to electroporation, de-beaded cells were centrifuged for 10 minutes at 90×g, aspirated, and resuspended in the Lonza electroporation buffer P3 at 20 μL buffer per one million cells. For optimal editing, one million T cells were electroporated per well using a Lonza 4D 96-well electroporation system with pulse code EH115. Alternate cell concentrations from 200,000 up to 2 million cells per well showed lower efficiencies. Alternate electroporation buffers were used as indicated, but had different optimal pulse settings (EO155 for OMEM buffer). Unless otherwise indicated, 2.5 μLs of RNPs (50 pmols total) were electroporated, along with 2 μLs of HDR Template at 2 μgs/μL (4 μgs HDR Template total).

For 96-well experiments, HDRTs were first aliquoted into wells of a 96-well polypropylene V-bottom plate. RNPs were then added to the HDRTs and allowed to incubate together at RT for at least 30 seconds. Finally, cells resuspended in electroporation buffer were added, briefly mixed by pipetting with the HDRT and RNP, and 24 μLs of total volume (cells+RNP+HDRT) was transferred into a 96 well electroporation cuvette plate. Immediately following electroporation, 80 μLs of pre-warmed media (without cytokines) was added to each well, and cells were allowed to rest for 15 minutes at 37° C. in a cell culture incubator while remaining in the electroporation cuvettes. After 15 minutes, cells were moved to final culture vessels.

### C-Terminal Modification of Endogenous Proteins

A general gene targeting strategy for inserting a fluorescent marker at the C-terminus of any desired protein was designed. Guide RNAs (gRNAs) that cut near the stop codon in the final exon of a gene as well as an HDR DNA template containing the sequence encoding the super folder green fluorescent protein (sfGFP) flanked by homology arms that each included roughly 300 base pairs of DNA homologous to the 300 base pairs of DNA upstream and downstream of the gRNA's cut site were made. The gRNA was complexed with Cas9 to form a ribonucleoprotein (RNP), and when the RNP and HDR DNA template were introduced into the cell via electroporation, the RNP cut near the stop codon and the HDR DNA Template was integrated at the cut site through homology directed repair (HDR).

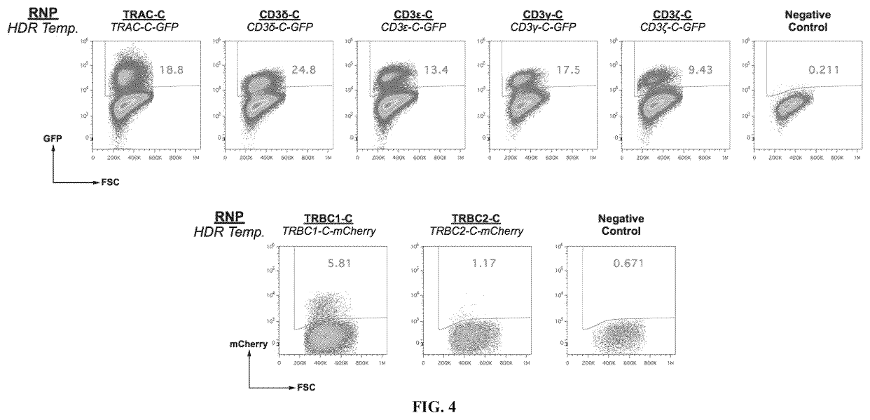

To test whether TCR complex proteins, i.e., protein members of the T-Cell Receptor (TCR) complex, could be modified at the C-terminus, each individual subunit of the TCR complex, namely the TCR alpha chain (TRAC), the TCR beta chain (TRBC), CD38 chain, CD3ε chain, CD3γ chain, and CD3ζ chain, was tagged with the fluorescent protein sfGFP or mCherry using the methodology described above. Human T-Cells were collected, edited, and analyzed using flow cytometry, and the results from flow cytometry analysis are depicted in FIG. 4. As evidenced in negative control samples, unedited bulk T-Cells do not naturally express GFP or mCherry. T-Cells electroporated with RNP and the corresponding HDR DNA Template express appreciable levels of GFP or mCherry.

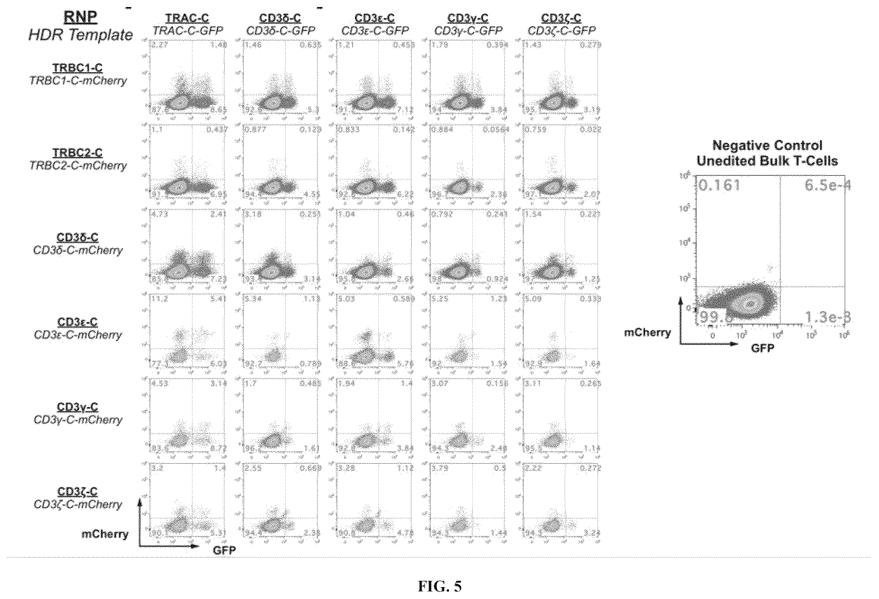

In addition to tagging individual members of the TCR complex with a fluorescence marker (FIG. 4), multiple components of the TCR complex were multiplexed and tagged with multiple fluorescence markers, simultaneously. Again, human T-Cells were collected, edited, and analyzed on the flow cytometer. In the matrix in FIG. 5, each sample received a unique set of two RNP and two HDR DNA Templates, which are denoted by the corresponding row and column labels. All multiplexed conditions yielded appreciable GFP+ populations, mCherry+ populations, and GFP+mCherry+ populations. Unedited bulk T-Cells analyzed using the same parameters did not fluoresce in the mCherry or GFP channel.

### Enhancement of TCR Signaling

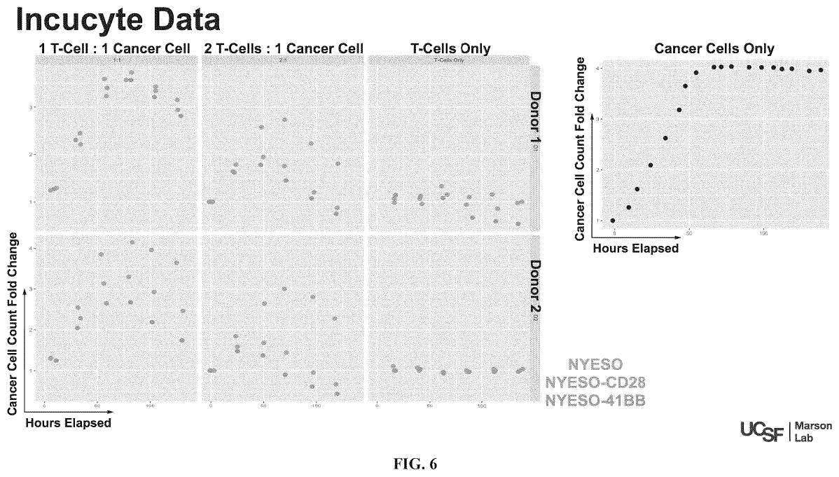

Endogenous co-stimulatory molecules generally act in conjunction with the T-Cell Receptor (TCR) to enhance TCR signaling by strengthening or activating additional signaling cascades involved in proliferation, preventing apoptosis, and/or increasing cytotoxic capacities. In naïve T-Cells, co-stimulation is critical for priming and ensuring that the antigen being targeted is non-self. In T-Cells with known and/or re-wired specificity, co-stimulatory signals can be utilized to enhance TCR signaling and T Cell function. Experiments were conducted to determine if adding a co-stimulatory molecule's cytoplasmic domain to the C-terminus or cytoplasmic tail of the TCR could enhance T cell function. To do this, a DNA construct (FIG. 6) containing the full length NY-ESO-1 TCR beta chain with the addition of the DNA sequence encoding either the cytoplasmic tail of the costimulatory molecule CD28 or the costimulatory molecule 4-1BB at its 3′ end, the VJ region of the NY-ESO-1 TCR's alpha chain, and homology arms was constructed. An exemplary nucleotide sequence for this construct is set forth herein as SEQ ID NO: 14. When electroporated along with a Cas9 RNP targeting TRAC Exon 1 into primary human T cells, the DNA construct was integrated into the TRAC gene locus via homology directed repair. Edited cells expressed an NY-ESO-1 specific TCR tagged with a co-stimulatory cytoplasmic domain under endogenous regulation.

### In Vitro Cancer Cell Killing by TCR Modification in Primary Human T Cells

To determine whether NY-ESO-1 specific T-Cells benefit from the addition of a co-stimulatory molecule's cytoplasmic tail on the TCR, an in vitro cancer cell killing assay was used to compare T cell function. DNA constructs containing the NY-ESO-1 TCR (NYESO), the NY-ESO-1 TCR with the cytoplasmic tail of CD28 fused to it (NYESO-CD28), or the NY-ESO-1 TCR with the cytoplasmic tail of 41BB fused to it (NYESO-41BB) were knocked-in to primary human T-Cells, and NY-ESO-1+ T-Cells were subsequently sorted and expanded from bulk edited samples. Following expansion, the NY-ESO-1 TCR T-Cell variants were co-cultured with the A375-RFP cancer cell line, which expresses the red fluorescence protein and the NY-ESO-1 tumor antigen. Cancer cell growth was measured by the Incucyte, which captures an image of each sample, analyzes the number of red fluorescent cancer cells currently present in each sample, and records its measurements at set intervals over the course of a week. Samples containing only cancer cells saw a sharp increase in cancer cell count followed by a plateau, as the well's carrying capacity was reached (FIG. 6) whereas samples containing only T cells, which do not express RFP, saw no measurement of cell growth (FIG. 6). In all four samples where A375-RFP cancer cells were co-cultured with NY-ESO-1 TCR+ T-Cells, cancer cell killing was observable. At the 2 T cell: 1 cancer cell ratio, NYESO-CD28 and NYESO-41BB clearly eliminate cancer cells with faster kinetics, and although the difference is not as marked, this trend applies to the 1 T cell: 1 cancer cell ratio.

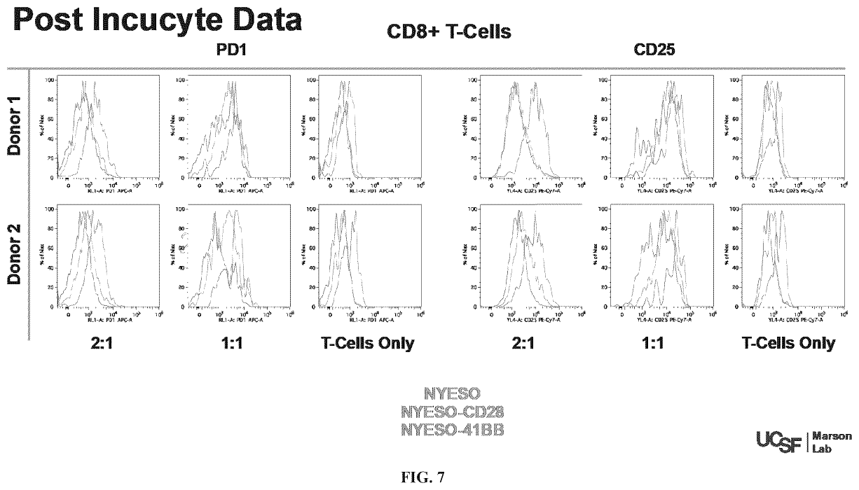

Following the in vitro killing assay, T cells were recovered and profiled by flow cytometry for expression patterns of a variety of cell surface markers to help discern any observed functional differences. In FIG. 7, it was observed that, in NYESO-CD28 and NYESO-41BB T cells PD1 expression levels trended lower at the end of the killing assay. Lower PD1 levels typically correlate with less T cell exhaustion, and may help explain the increased potency of the NYESO-CD28 and NYESO-41BB cells in eliminating cancer cells. It is likely that the NYESO-CD28 and NYESO-41BB cells are either clearing cancer cells at such a quick rate that they are not becoming exhausted or that the addition of the co-stimulatory cytoplasmic domain has activated signaling cascades that resulted in an altered cell state less prone to exhaustion. In FIG. 7, it was also observed that NYESO-CD28 and NYESO-41BB T cells also trended toward lower CD25 expression levels.

### Example 2

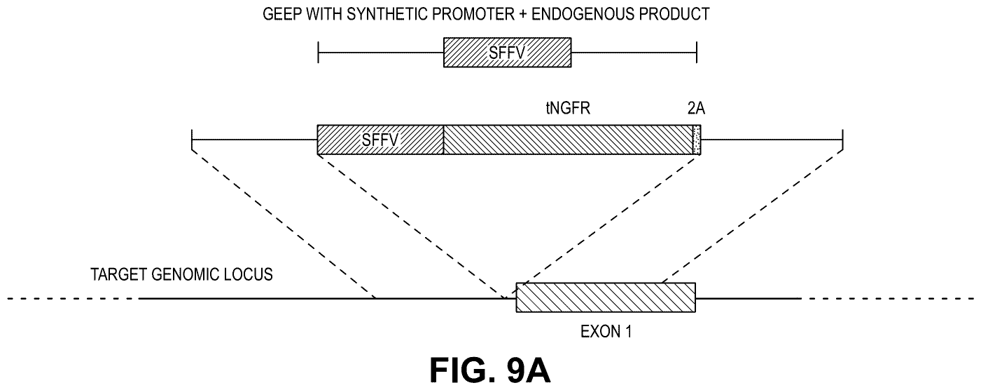

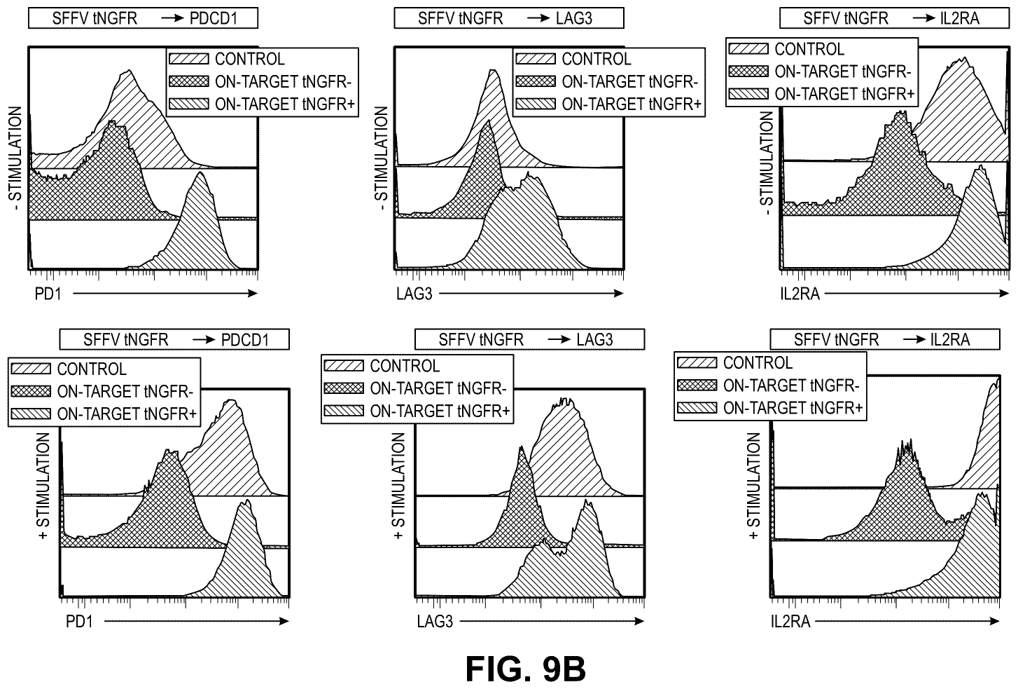

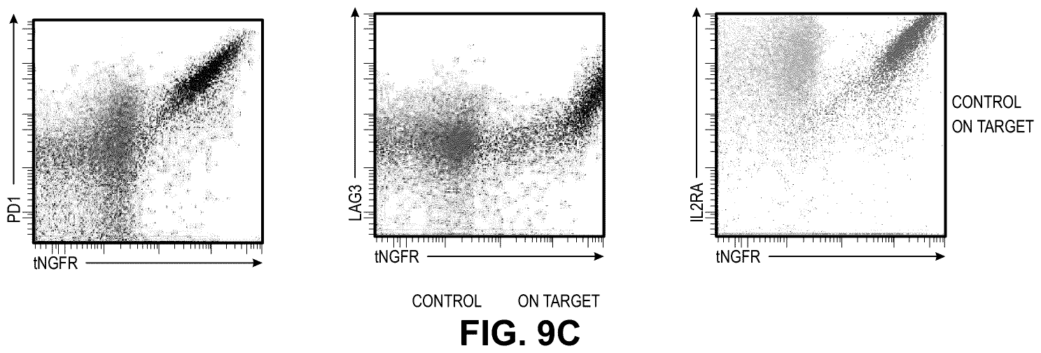

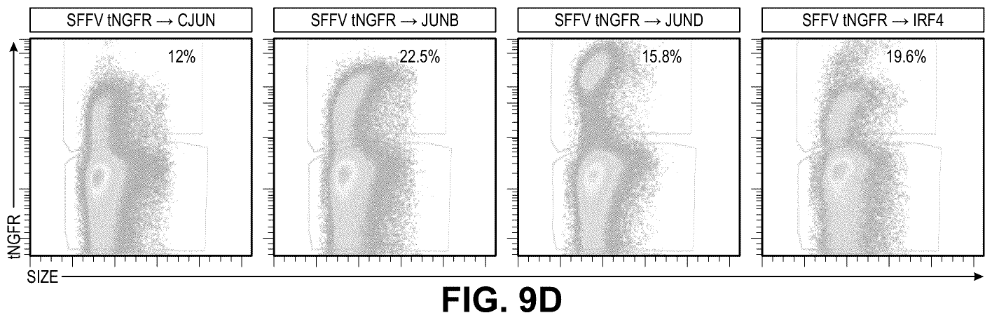

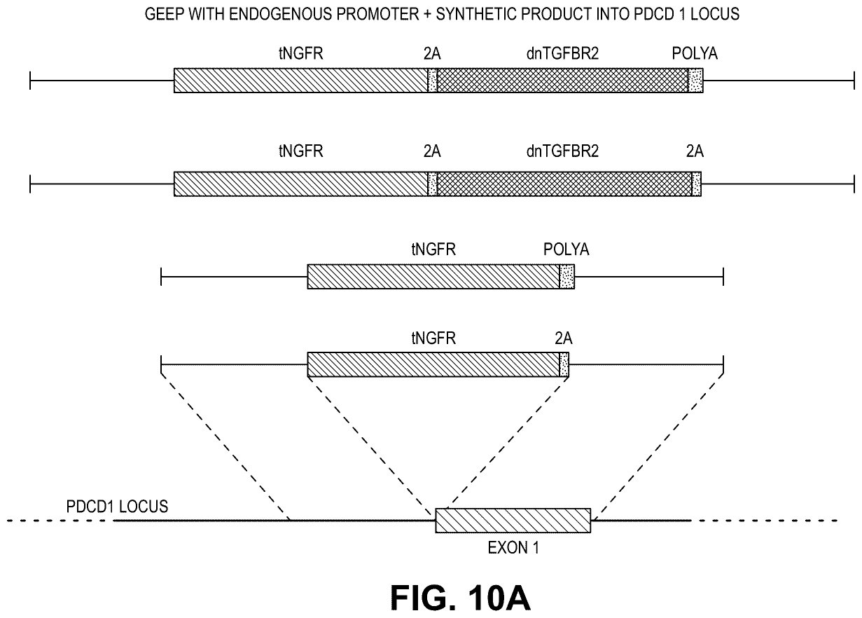

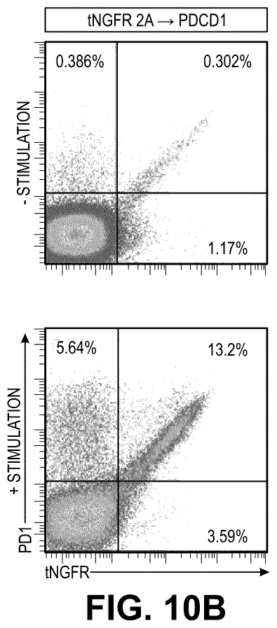

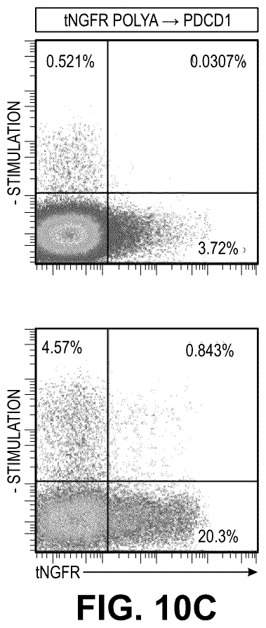

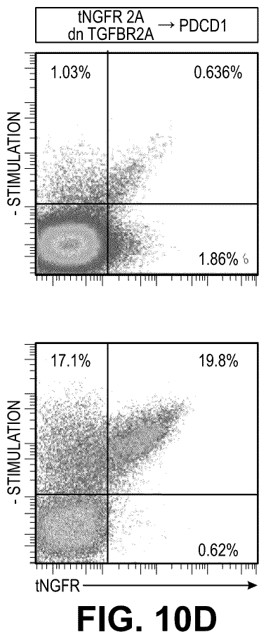

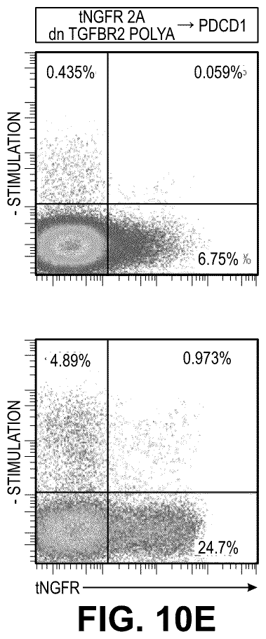

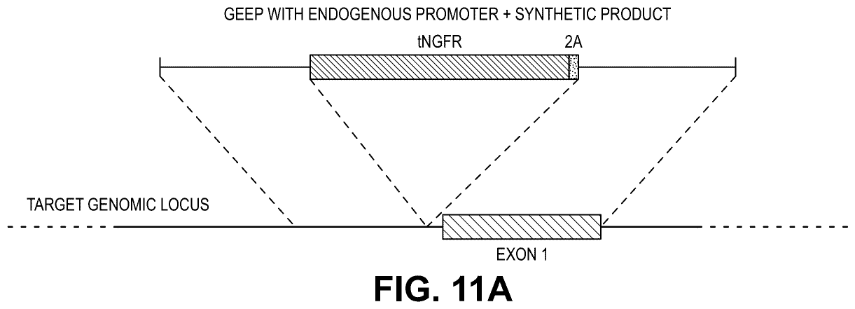

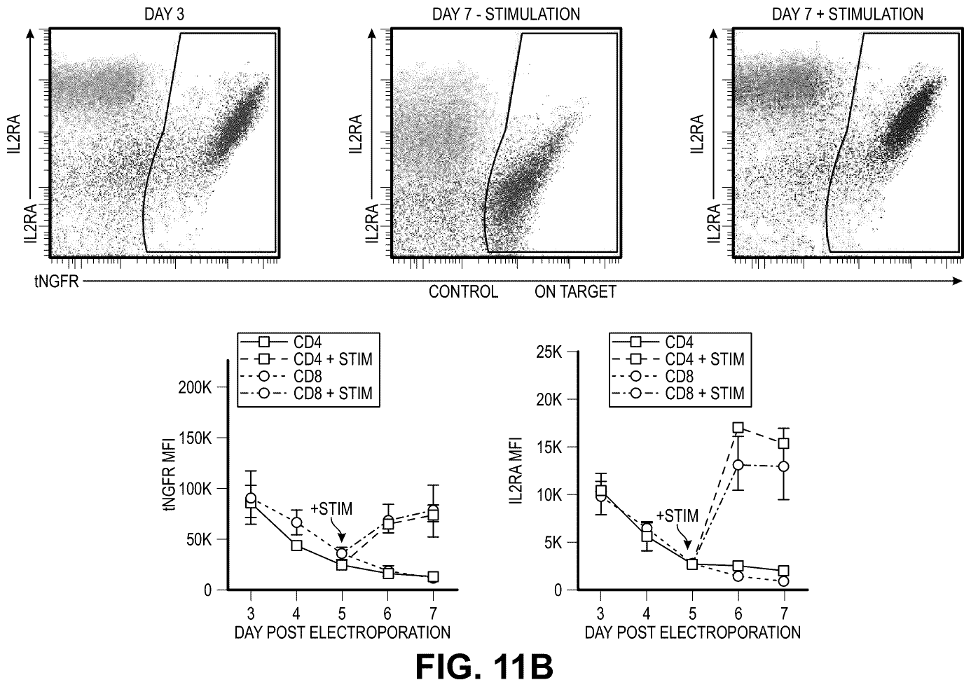

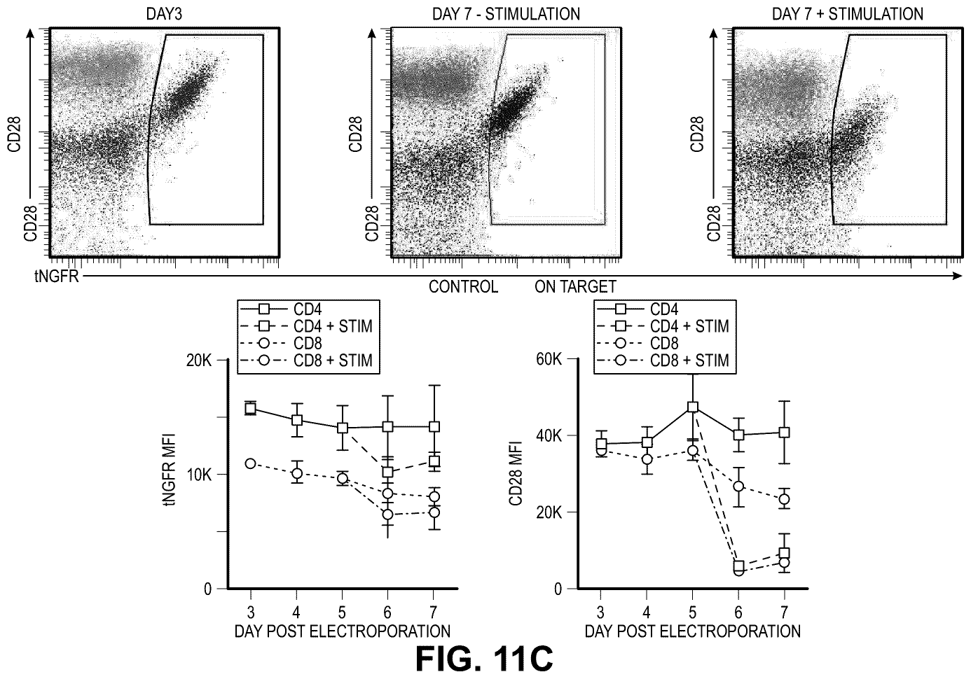

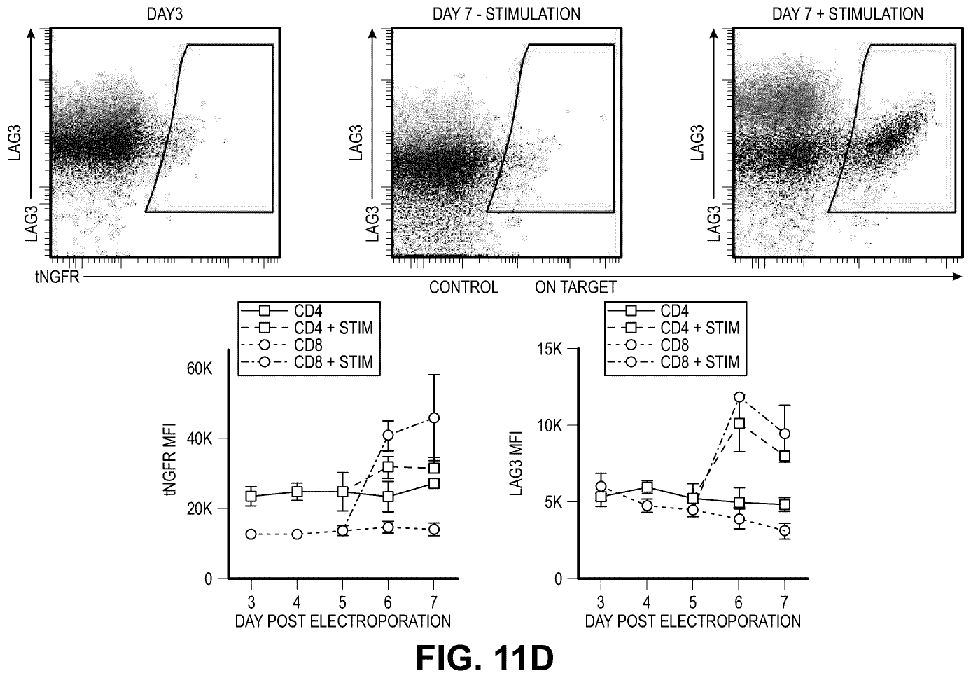

Integration of a new viral promoter to the transcriptional start site of an endogenous gene creates a ‘promoter GEEP’ with a synthetic promoter driving expression of an endogenous gene product (FIG. 8B). Promoter GEEPs at IL2RA and PDCD1 showed continuing high expression of IL2RA and PD1 in resting cells 9 days after TCR stimulation, whereas the endogenous regulatory circuit for these activation-dependent genes showed low expression levels (FIG. 8B and FIG. 9). In contrast, integration of a new gene product at the same site creates a ‘product GEEP’ with an endogenous regulatory circuit driving expression of a new synthetic gene product (FIG. 8C). We created product GEEPs at the PDCD1 locus containing either a 2A peptide to maintain expression of the endogenous PD1 gene or a poly A sequence to remove endogenous PD1 gene expression (FIG. 8C and FIG. 10). Product GEEPs created at the IL2RA, CD28, and LAG3 loci all mirrored the expression dynamics of their respective endogenous genes (FIG. 8D and FIG. 11). Integration of a new extracellular domain specifically in front of a target surface receptors transmembrane domain creates a ‘specificity GEEP’ with a synthetic specificity driving endogenous signaling (FIG. 8E), such as at the endogenous TCRα locus where we have previously reported the ability to replace the extracellular TCR specificity while maintaining the endogenous constant signaling domains. Finally, integration of a new signaling domain to a surface receptor after the transmembrane domain creates a ‘signaling GEEP’ where an endogenous specificity drives synthetic signaling (FIG. 8F). Signaling GEEPs were created at all four CD3 gene loci (CD3D, CD3E, CD3G, CD3Z) with either a CD28 intraceullar domain or a 41BB intracellular domain appended (FIG. 12). While none of the CD28 intracellular domain fusions showed increased proliferation in the presence of CD3 stimulation in comparison to control knockin cells (FIG. 12), a CD3ζ-41BB signaling GEEP specifically showed increased proliferation in response to CD3 stimulation in the absence of CD28 costimulation (FIG. 8G).

### Methods

### Isolation of Human Primary T Cells for Gene Targeting

Primary human T cells were isolated from either fresh whole blood or residuals from leukoreduction chambers after Trima Apheresis (Blood Centers of the Pacific) from healthy donors. Peripheral blood mononuclear cells (PBMCs) were isolated from whole blood samples by Ficoll centrifugation using SepMate tubes (STEMCELL, per manufacturer's instructions). T cells were isolated from PBMCs from all cell sources by magnetic negative selection using an EasySep Human T Cell Isolation Kit (STEMCELL, per manufacturer's instructions). Isolated T cells were either used immediately following isolation for electroporation experiments or frozen down in Bambanker freezing medium (Bulldog Bio) per manufacturer's instructions for later use. Freshly isolated T cells were stimulated as described below. Previously frozen T cells were thawed, cultured in media without stimulation for 1 day, and then stimulated and handled as described for freshly isolated samples. Fresh blood was taken from healthy human donors under a protocol approved by the UCSF Committee on Human Research (CHR #13-11950).

### Primary Human T Cell Culture

XVivo15 medium (STEMCELL) supplemented with 5% fetal bovine serum, 50 uM 2-mercaptoethanol, and 10 uM N-acetyl L-cystine was used to culture primary human T cells. In preparation for electroporation, T cells were stimulated for 2 days at a starting density of approximately 1 million cells per mL of media with anti-human CD3/CD28 magnetic Dynabeads (ThermoFisher), at a bead to cell ratio of 1:1, and cultured in XVivo15 media containing IL-2 (500 U ml-1; UCSF Pharmacy), IL-7 (5 ng ml-1; ThermoFisher), and IL-15 (5 ng ml-1; Life Tech). Following electroporation, T cells were cultured in XVivo15 media containing IL-2 (500 U ml-1) and maintained at approximately 1 million cells per mL of media. Every 2-3 days, electroporated T cells were topped up, with or without splitting, with additional media along with additional fresh IL-2 (final concentration of 500 U ml-1). When necessary, T cells were transferred to larger culture vessels.

### RNP Production

RNPs were produced by complexing a two-component gRNA to Cas9. The two-component gRNA consisted of a crRNA and a tracrRNA, both chemically synthesized (Dharmacon, IDT) and lyophilized. Upon arrival, lyophilized RNA was resuspended in 10 mM Tris-HCL (7.4 pH) with 150 mM KCl at a concentration of 160 uM and stored in aliquots at −80° C. Cas9-NLS (QB3 Macrolab) was recombinantly produced, purified, and stored at 40 uM in 20 mM HEPES-KOH, pH 7.5, 150 mM KCl, 10% glycerol, 1 mM DTT.

To produce RNPs, the crRNA and tracrRNA aliquots were thawed, mixed 1:1 by volume, and annealed by incubation at 37° C. for 30 min to form an 80 uM gRNA solution. Next, the gRNA solution was mixed 1:1 by volume with Cas9-NLS (2:1 gRNA to Cas9 molar ratio) and incubated at 37° C. for 15 min to form a 20 uM RNP solution. RNPs were electroporated immediately after complexing.

### Double-Stranded HDR DNA Template Production

Each double-stranded homology directed repair DNA template (HDRT) contained a novel/synthetic DNA insert flanked by homology arms. We used Gibson Assemblies to construct plasmids containing the HDRT and then used these plasmids as templates for high-output PCR amplification (Kapa Hot Start polymerase). The resulting PCR amplicons/HDRTs were SPRI purified (1.0×) and eluted into H2O. The concentrations of eluted HDRTs were determined, using a 1:20 dilution, by NanoDrop and then normalized to 1 μg/μL. The size of the amplified HDRT was confirmed by gel electrophoresis in a 1.0% agarose gel.

The following sequences were used for the HDR template:

### CD3Zeta-41BB HDR DNA Template

TATGGGAGGTGGGAGACTCCTTTCTCTTAGGTCCAGCAGGAAGTTGGCG
GGGCCCAAGCACTGTAAGGCACAGCATTTGAGGAGCTGAGAAGAGGGGT
GAGAATTTAGCTGGAAAGGAGTTGCTGCAAGGCCATTCCCGGCAGGGCA
CAGCACCCATCTACCAACGAAGCTGTTGCAGCCAAGGCTCCTGCCCGTG
GGGCCAGGGGGATTATTCCTGGGCCTCTGGAGGCTGGGTGGGTGGTCAC
AGGGCTGTGCTGCAGAGACACCTGTTGGCCTCTGGGTTGGCTCCTGCCC
ACACAGGCTACTGACCCACTCTTTGTTTTCTGATTTGCTTTCACGCCAG
GGTCTCAGTACAGCCACCAAGGACACCTACGACGCCCTTCACATGCAGG
CCCTGCCCCCTCGCGGATCGGGTGGGACTAGTGGCaaacgcggccgcaa
aaaactgctgtatatttttaaacagccgtttatgcgcccggtgcagacc
acccaggaagaagatggctgcagctgccgctttccggaagaagaagaag
gcggctgcgaactgCGGGCCAAGCGGTCCGGATCCGGAGCCACCAACTT
CAGCCTGCTGAAGCAGGCCGGCGACGTGGAGGAGAACCCCGGCCCCATG
gtgagcaagggcgaggaggataacatggccatcatcaaggagttcatgc
gcttcaaggtgcacatggagggctccgtgaacggccacgagttcgagat
cgagggcgagggcgagggccgcccctacgagggcacccagaccgccaag
ctgaaggtgaccaagggtggccccctgcccttcgcctgggacatcctgt
cccctcagttcatgtacggctccaaggcctacgtgaagcaccccgccga
catccccgactacttgaagctgtccttccccgagggcttcaagtgggag
cgcgtgatgaacttcgaggacggcggcgtggtgaccgtgacccaggact
cctccctgcaggacggcgagttcatctacaaggtgaagctgcgcggcac
caacttcccctccgacggccccgtaatgcagaagaagaccatgggctgg
gaggcctcctccgagcggatgtaccccgaggacggcgccctgaagggcg
agatcaagcagaggctgaagctgaaggacggcggccactacgacgctga
ggtcaagaccacctacaaggccaagaagcccgtgcagctgcccggcgcc
tacaacgtcaacatcaagttggacatcacctcccacaacgaggactaca
ccatcgtggaacagtacgaacgcgccgagggccgccactccaccggcgg
catggacgagctgtacaagggaaccggtGCTggaagtggtTAACAGCCA
GGAGATTTCACCACTCAAAGGCCAGACCTGCAGACGCCCAGATTATGAG
ACACAGGATGAAGCATTTACAACCCGGTTCACTCTTCTCAGCCACTGAA
GTATTCCCCTTTATGTACAGGATGCTTTGGTTATATTTAGCTCCAAACC
TTCACACACAGACTGTTGTCCCTGCACTCTTTAAGGGAGTGTACTCCCA
GGGCTTACGGCCCTGGCCTTGGGCCCTCTGGTTTGCCGGTGGTGCAGGT
AGACCTGTCTCCTGGCGGTTCCTCGTTCTCCCTGGGAGGCGGGCGCACT
GCCTCTCACAGCTGAGTTGTTGAGTCTGTTTTGTAAAGTCCCCAGAGAA
AGCGCA

CD3Zeta-41BB HDR DNA Template w/Shuttle Sequences (Shuttle Sequences Italicized and Underlined)

TGGCGGGACTAGTGGCTGAGCCTCGCTAACAGCCAGCGGTATGGGAGGT
GGGAGACTCCTTTCTCTTAGGTCCAGCAGGAAGTTGGCGGGGCCCAAGC
ACTGTAAGGCACAGCATTTGAGGAGCTGAGAAGAGGGGTGAGAATTTAG
CTGGAAAGGAGTTGCTGCAAGGCCATTCCCGGCAGGGCACAGCACCCAT
CTACCAACGAAGCTGTTGCAGCCAAGGCTCCTGCCCGTGGGGCCAGGGG
GATTATTCCTGGGCCTCTGGAGGCTGGGTGGGTGGTCACAGGGCTGTGC
TGCAGAGACACCTGTTGGCCTCTGGGTTGGCTCCTGCCCACACAGGCTA
CTGACCCACTCTTTGTTTTCTGATTTGCTTTCACGCCAGGGTCTCAGTA
CAGCCACCAAGGACACCTACGACGCCCTTCACATGCAGGCCCTGCCCCC
TCGCGGATCGGGTGGGACTAGTGGCaaacgcggccgcaaaaaactgctg
tatatttttaaacagccgtttatgcgcccggtgcagaccacccaggaag
aagatggctgcagctgccgctttccggaagaagaagaaggcggctgcga
actgCGGGCCAAGCGGTCCGGATCCGGAGCCACCAACTTCAGCCTGCTG
AAGCAGGCCGGCGACGTGGAGGAGAACCCCGGCCCCATGgtgagcaagg
gcgaggaggataacatggccatcatcaaggagttcatgcgcttcaaggt
gcacatggagggctccgtgaacggccacgagttcgagatcgagggcgag
ggcgagggccgcccctacgagggcacccagaccgccaagctgaaggtga
ccaagggtggccccctgcccttcgcctgggacatcctgtcccctcagtt
catgtacggctccaaggcctacgtgaagcaccccgccgacatccccgac
tacttgaagctgtccttccccgagggcttcaagtgggagcgcgtgatga
acttcgaggacggcggcgtggtgaccgtgacccaggactcctccctgca
ggacggcgagttcatctacaaggtgaagctgcgcggcaccaacttcccc
tccgacggccccgtaatgcagaagaagaccatgggctgggaggcctcct
ccgagcggatgtaccccgaggacggcgccctgaagggcgagatcaagca
gaggctgaagctgaaggacggcggccactacgacgctgaggtcaagacc
acctacaaggccaagaagcccgtgcagctgcccggcgcctacaacgtca
acatcaagttggacatcacctcccacaacgaggactacaccatcgtgga
acagtacgaacgcgccgagggccgccactccaccggcggcatggacgag
ctgtacaagggaaccggtGCTggaagtggtTAACAGCCAGGAGATTTCA
CCACTCAAAGGCCAGACCTGCAGACGCCCAGATTATGAGACACAGGATG
AAGCATTTACAACCCGGTTCACTCTTCTCAGCCACTGAAGTATTCCCCT
TTATGTACAGGATGCTTTGGTTATATTTAGCTCCAAACCTTCACACACA
GACTGTTGTCCCTGCACTCTTTAAGGGAGTGTACTCCCAGGGCTTACGG
CCCTGGCCTTGGGCCCTCTGGTTTGCCGGTGGTGCAGGTAGACCTGTCT
CCTGGCGGTTCCTCGTTCTCCCTGGGAGGCGGGCGCACTGCCTCTCACA
GCTGAGTTGTTGAGTCTGTTTTGTAAAGTCCCCAGAGAAAGCGCACCGC
TGGCTGTTAGCGAGGCTCAggtGCTggaagtggtG

### Primary T Cell Electroporation

For all electroporation experiments, primary T cells were prepared and cultured as described above. After stimulation for 48-56 hours, T cells were collected from their culture vessels and the anti-CD3/anti-CD28 Dynabeads were magnetically separated from the T cells. Immediately before electroporation, de-beaded cells were centrifuged for 10 min at 90 g, aspirated, and resuspended in the Lonza electroporation buffer P3. Each experimental condition received a range of 750,000-1 million activated T cells resuspended in 20 μL of P3 buffer, and all electroporation experiments were carried out in 96 well format.

For GEEPs knockins (FIG. 8), truncated Cas9 Target Sequences (tCTS) were additionally added to the 5′ and 3′ ends of the HDR template enabling a Cas9 ‘shuttle’ as described. For all variations, T cells resuspended in the electroporation buffer were added to the RNP and HDRT mixture, briefly mixed, and then transferred into a 96-well electroporation cuvette plate

All electroporations were done using a Lonza 4D 96-well electroporation system with pulse code EH115. Unless otherwise indicated, 3.5 μl RNPs (comprising 50 pmol of total RNP) were electroporated, along with 1-3 μl HDR Template at 1 μg μl-1 (1-3 μg HDR template total). Immediately after all electroporations, 80 μl of pre-warmed media (without cytokines) was added to each well, and cells were allowed to rest for 15 min at 37° C. in a cell culture incubator while remaining in the electroporation cuvettes. After 15 min, cells were moved to final culture vessels.

### Synthetic Product+Endogenous Product Kinetics Flow Cytometry Analysis

Non-virally edited T-cells were split into multiple replicates and analyzed by flow cytometry every day for a 5-day period starting on Day 3 after electroporation. During that 5-day period, T-cells were topped up every 2 days with additional media and IL-2, to a final concentration of 500 U/mL, with or without a 1:1 split. At Day 5 post electroporation, one set of cells was stimulated with CD3/CD28 Dynabeads and the other was left unstimulated.

### In Vitro Proliferation Assay

Non-virally edited T-cells were expanded in independent cultures prior to the assay. The unsorted, edited populations were pooled after approximately two weeks of expansion (with 500 U/mL of IL-2 supplemented every 2-3 days) for a competitive mixed proliferation assay.

For the CD3 competitive mixed proliferation assay, we pooled unsorted samples with CD28IC-2A-GFP, 41BBIC-2A-mCherry, or 2A-BFP knocked-in to the same CD3 complex member's gene locus. To determine the input numbers for pooling, we took into account the number of viable GFP+, mCherry+, or BFP+ in the respective populations (knock-in %*total viable cell count), as determined by flow cytometry analysis. The pooled sample was then distributed into round bottom 96 well plates at a starting total cell count of 50,000. The distributed samples were then cultured without stimulation, with CD3 stimulation only, with CD28 stimulation only, or with CD3/CD28 stimulation. CD3 and/or CD28 stimulation was done with plate bound antibodies. All samples were cultured in XVivo15 media supplemented with IL-2 (50 U/mL). After 4 days in culture, samples were analyzed by flow cytometry for relative outgrowth of GFP+ and mCherry+ subpopulations relative to the BFP+ subpopulation.

The sequences for the CD3ζ-1-B44 fusion are:

### Edited Exon 8:

GGTCTCAGTACAGCCACCAAGGACACCTACGACGCCCTTCACATGCAGG
CCCTGCCCCCTCGCGGATCGGGTGGGACTAGTGGCaaacgcggccgcaa
aaaactgctgtatatttttaaacagccgtttatgcgcccggtgcagacc
acccaggaagaagatggctgcagctgccgctttccggaagaagaagaag
gcggctgcgaactgCGGGCCAAGCGGTCCGGATCCGGAGCCACCAACTT
CAGCCTGCTGAAGCAGGCCGGCGACGTGGAGGAGAACCCCGGCCCCATG
gtgagcaagggcgaggaggataacatggccatcatcaaggagttcatgc
gcttcaaggtgcacatggagggctccgtgaacggccacgagttcgagat
cgagggcgagggcgagggccgcccctacgagggcacccagaccgccaag
ctgaaggtgaccaagggtggccccctgcccttcgcctgggacatcctgt
cccctcagttcatgtacggctccaaggcctacgtgaagcaccccgccga
catccccgactacttgaagctgtccttccccgagggcttcaagtgggag
cgcgtgatgaacttcgaggacggcggcgtggtgaccgtgacccaggact
cctccctgcaggacggcgagttcatctacaaggtgaagctgcgcggcac
caacttcccctccgacggccccgtaatgcagaagaagaccatgggctgg
gaggcctcctccgagcggatgtaccccgaggacggcgccctgaagggcg
agatcaagcagaggctgaagctgaaggacggcggccactacgacgctga
ggtcaagaccacctacaaggccaagaagcccgtgcagctgcccggcgcc
tacaacgtcaacatcaagttggacatcacctcccacaacgaggactaca
ccatcgtggaacagtacgaacgcgccgagggccgccactccaccggcgg
catggacgagctgtacaagggaaccggtGCTggaagtggtTAA

Edited cDNA:

(SEQ ID NO: 18)
ATGAAGTGGAAGGCGCTTTTCACCGCGGCCATCCTGCAGGCACAGTTGC
CGATTACAGAGGCACAGAGCTTTGGCCTGCTGGATCCCAAACTCTGCTA
CCTGCTGGATGGAATCCTCTTCATCTATGGTGTCATTCTCACTGCCTTG
TTCCTGAGAGTGAAGTTCAGCAGGAGCGCAGACGCCCCCGCGTACCAGC
AGGGCCAGAACCAGCTCTATAACGAGCTCAATCTAGGACGAAGAGAGGA
GTACGATGTTTTGGACAAGAGACGTGGCCGGGACCCTGAGATGGGGGGA
AAGCCGCAGAGAAGGAAGAACCCTCAGGAAGGCCTGTACAATGAACTGC
AGAAAGATAAGATGGCGGAGGCCTACAGTGAGATTGGGATGAAAGGCGA
GCGCCGGAGGGGCAAGGGGCACGATGGCCTTTACCAGGGTCTCAGTACA
GCCACCAAGGACACCTACGACGCCCTTCACATGCAGGCCCTGCCCCCTC
GCGGTCTCAGTACAGCCACCAAGGACACCTACGACGCCCTTCACATGCA
GGCCCTGCCCCCTCGCGGATCGGGTGGGACTAGTGGCaaacgcggccgc
aaaaaactgctgtatatttttaaacagccgtttatgcgcccggtgcaga
ccacccaggaagaagatggctgcagctgccgctttccggaagaagaaga
aggcggctgcgaactgCGGGCCAAGCGGTCCGGATCCGGAGCCACCAAC
TTCAGCCTGCTGAAGCAGGCCGGCGACGTGGAGGAGAACCCCGGCCCCA
TGgtgagcaagggcgaggaggataacatggccatcatcaaggagttcat
gcgcttcaaggtgcacatggagggctccgtgaacggccacgagttcgag
atcgagggcgagggcgagggccgcccctacgagggcacccagaccgcca
agctgaaggtgaccaagggggccccctgcccttcgcctgggacatcctg
tcccctcagttcatgtacggctccaaggcctacgtgaagcaccccgccg
acatccccgactacttgaagctgtccttccccgagggcttcaagtggga
gcgcgtgatgaacttcgaggacggcggcgtggtgaccgtgacccaggac
tcctccctgcaggacggcgagttcatctacaaggtgaagctgcgcggca
ccaacttcccctccgacggccccgtaatgcagaagaagaccatgggctg
ggaggcctcctccgagcggatgtaccccgaggacggcgccctgaagggc
gagatcaagcagaggctgaagctgaaggacggcggccactacgacgctg
aggtcaagaccacctacaaggccaagaagcccgtgcagctgcccggcgc
ctacaacgtcaacatcaagttggacatcacctcccacaacgaggactac
accatcgtggaacagtacgaacgcgccgagggccgccactccaccggcg
gcatggacgagctgtacaagggaaccggtGCTggaagtggtTAA

### Edited Full-Length Amino Acid:

(SEQ ID NO: 19)
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPRGSGGTSGKRGRKKLLYIFKQPFMRPVQTTQEE
DGCSCRFPEEEEGGCELRAKRSGSGATNFSLLKQAGDVEENPGPMVSKG
EEDNMAIIKEFMRFKVHMEGSVNGHEFEIEGEGEGRPYEGTQTAKLKVT
KGGPLPFAWDILSPQFMYGSKAYVKHPADIPDYLKLSFPEGFKWERVMN
FEDGGVVTVTQDSSLQDGEFIYKVKLRGTNFPSDGPVMQKKTMGWEASS
ERMYPEDGALKGEIKQRLKLKDGGHYDAEVKTTYKAKKPVQLPGAYNVN
IKLDITSHNEDYTIVEQYERAEGRHSTGGMDELYKGTGAGSG*.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Sequences

SEQ ID NO: 1
GSGGTSGRSKRSRLLHSDYMNMTPRRPGPTRKHYQPYAPPRDFAAYRS
SEQ ID NO: 2
GSGGTSGKRGRKKLLYIFKQPFMRPVQTTQEEDGCSCRFPEEEEGGCEL
SEQ ID NO: 3
GSGGTSGMDFEYLEIRQLETQADPTGRLLDAWQGRPGASVGRLLELLTKLGRDDVL
LELGPSIEEDCQKYILKQQQEEAEKPLQVAAVDSSVPRTAELAGITTLDDPLG
SEQ ID NO: 4
GSGGTSGLCARPRRSPAQEDGKVYINMPGRG
SEQ ID NO: 5
RAKR
SEQ ID NO: 6
GSGGTSGRSKRSRLLHSDYMNMTPRRPGPTRKHYQPYAPPRDFAAYRSRAKRSGSG
SEQ ID NO: 7
GSGGTSGKRGRKKLLYIFKQPFMRPVQTTQEEDGCSCRFPEEEEGGCELRAKRSGSG
SEQ ID NO: 8
GSGGTSGMDFEYLEIRQLETQADPTGRLLDAWQGRPGASVGRLLELLTKLGRDDVL
LELGPSIEEDCQKYILKQQQEEAEKPLQVAAVDSSVPRTAELAGITTLDDPLGRAKRS
GSG
SEQ ID NO: 9
GSGGTSGLCARPRRSPAQEDGKVYINMPGRGRAKRSGSG
SEQ ID NO: 10
GGATCGGGTGGGACTAGTGGCcgcagcaaacgcagccgcctgctgcatagcgattatatgaacatgACTccg
AGAAGAccgGGAccgacccgcaaacattatcagccgtatgcgccgccgcgcgattttgcggcgtatcgcagcCGGGC
CAAGCGGTCCGGATCCGGA
SEQ ID NO: 11
GGATCGGGTGGGACTAGTGGCaaacgcggccgcaaaaaactgctgtatatttttaaacagccgtttatgcgcccgg
tgcagaccacccaggaagaagatggctgcagctgccgctttccggaagaagaagaaggcggctgcgaactgCGGGCCAA
GCGGTCCGGATCCGGA
SEQ ID NO: 12
GGATCGGGTGGGACTAGTGGCATGGACTTTGAGTACTTGGAGATCCGGCAACTG
GAGACACAAGCGGACCCCACTGGCAGGCTGCTGGACGCCTGGCAGGGACGCCCT
GGCGCCTCTGTAGGCCGACTGCTCGAGCTGCTTACCAAGCTGGGCCGCGACGAC
GTGCTGCTGGAGCTGGGACCCAGCATTGAGGAGGATTGCCAAAAGTATATCTTG
AAGCAGCAGCAGGAGGAGGCTGAGAAGCCTTTACAGGTGGCCGCTGTAGACAGC
AGTGTCCCACGGACAGCAGAGCTGGCGGGCATCACCACACTTGATGACCCCCTG
GGGCGGGCCAAGCGGTCCGGATCCGGA
SEQ ID NO: 13
GGATCGGGTGGGACTAGTGGCCTGTGCGCACGCCCACGCCGCAGCCCCGCCCAA
GAAGATGGCAAAGTCTACATCAACATGCCAGGCAGGGGCCGGGCCAAGCGGTCC
GGATCCGGA
SEQ ID NO: 14
TTTCAGGTTTCCTTGAGTGGCAGGCCAGGCCTGGCCGTGAACGTTCACTGAAATC
ATGGCCTCTTGGCCAAGATTGATAGCTTGTGCCTGTCCCTGAGTCCCAGTCCATC
ACGAGCAGCTGGTTTCTAAGATGCTATTTCCCGTATAAAGCATGAGACCGTGACT
TGCCAGCCCCACAGAGCCCCGCCCTTGTCCATCACTGGCATCTGGACTCCAGCCT
GGGTTGGGGCAAAGAGGGAAATGAGATCATGTCCTAACCCTGATCCTCTTGTCCC
ACAGATATCCAGAACCCTGACCCTGCCTCCGGATCCGGAGAGGGCAGGGGATCT
CTCCTTACTTGTGGCGACGTGGAGGAGAACCCCGGCCCCATGAGCATCGGCCTCC
TGTGCTGTGCAGCCTTGTCTCTCCTGTGGGCAGGTCCAGTGAATGCTGGTGTCAC
TCAGACCCCAAAATTCCAGGTCCTGAAGACAGGACAGAGCATGACACTGCAGTG
TGCCCAGGATATGAACCATGAATACATGTCCTGGTATCGACAAGACCCAGGCAT
GGGGCTGAGGCTGATTCATTACTCAGTTGGTGCTGGTATCACTGACCAAGGAGA
AGTCCCCAATGGCTACAATGTCTCCAGATCAACCACAGAGGATTTCCCGCTCAGG
CTGCTGTCGGCTGCTCCCTCCCAGACATCTGTGTACTTCTGTGCCAGCAGTTACGT
CGGGAACACCGGGGAGCTGTTTTTTGGAGAAGGCTCTAGGCTGACCGTACTGGA
GGACCTGAAAAACGTGTTCCCACCCGAGGTCGCTGTGTTTGAGCCATCAGAAGC
AGAGATCTCCCACACCCAAAAGGCCACACTGGTATGCCTGGCCACAGGCTTCTA
CCCCGACCACGTGGAGCTGAGCTGGTGGGTGAATGGGAAGGAGGTGCACAGTGG
GGTCAGCACAGACCCGCAGCCCCTCAAGGAGCAGCCCGCCCTCAATGACTCCAG
ATACTGCCTGAGCAGCCGCCTGAGGGTCTCGGCCACCTTCTGGCAGAACCCCCGC
AACCACTTCCGCTGTCAAGTCCAGTTCTACGGGCTCTCGGAGAATGACGAGTGGA
CCCAGGATAGGGCCAAACCCGTCACCCAGATCGTCAGCGCCGAGGCCTGGGGTA
GAGCAGACTGTGGCTTCACCTCCGAGTCTTACCAGCAAGGGGTCCTGTCTGCCAC
CATCCTCTATGAGATCTTGCTAGGGAAGGCCACCTTGTATGCCGTGCTGGTCAGT
GCCCTCGTGCTGATGGCTATGGTCAAGAGAAAGGATTCCAGAGGCGGATCGGGT
GGGACTAGTGGCcgcagcaaacgcagccgcctgctgcatagcgattatatgaacatgACTccgAGAAGAccgG
GAccgacccgcaaacattatcagccgtatgcgccgccgcgcgattttgcggcgtatcgcagcCGGGCCAAGCGGTCC
GGATCCGGAGCCACCAACTTCAGCCTGCTGAAGCAGGCCGGCGACGTGGAGGAG
AACCCCGGCCCCATGGAGACCCTCTTGGGCCTGCTTATCCTTTGGCTGCAGCTGC
AATGGGTGAGCAGCAAACAGGAGGTGACGCAGATTCCTGCAGCTCTGAGTGTCC
CAGAAGGAGAAAACTTGGTTCTCAACTGCAGTTTCACTGATAGCGCTATTTACAA
CCTCCAGTGGTTTAGGCAGGACCCTGGGAAAGGTCTCACATCTCTGTTGCTTATT
CAGTCAAGTCAGAGAGAGCAAACAAGTGGAAGACTTAATGCCTCGCTGGATAAA
TCATCAGGACGTAGTACTTTATACATTGCAGCTTCTCAGCCTGGTGACTCAGCCA
CCTACCTCTGTGCTGTGAGGCCCCTGTACGGAGGAAGCTACATACCTACATTTGG
AAGAGGAACCAGCCTTATTGTTCATCCGTATATCCAGAACCCTGACCCTGCGGTG
TACCAGCTGAGAGACTCTAAATCCAGTGACAAGTCTGTCTGCCTATTCACCGATT
TTGATTCTCAAACAAATGTGTCACAAAGTAAGGATTCTGATGTGTATATCACAGA
CAAAACTGTGCTAGACATGAGGTCTATGGACTTCAAGAGCAACAGTGCTGTGGC
CTGGAGCAACAAATCTGACTTTGCATGTGCAAACGCCTTCAACAACAGCATTATT
CCAGAAGACACCTTCTTCCCCAGCCCAGGTAAGGGCAGCTTTGGTGCCTTCGCAG
GCTGTTTCCTTGCTTCAGGAATGGCCA
SEQ ID NO: 15
KRGRKKLLYIFKQPFMRPVQTTQEEDGCSCRFPEEEEGGCEL
SEQ ID NO: 16
RAKRSGSG
SEQ ID NO: 17
MKWKALFTAAILQAQLPITEAQSFGLLDPKLCYLLDGILFIYGVILTALFLRVKFSRSA
DAPAYQQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPRGSGGT
SGKRGRKKLLYIFKQPFMRPVQTTQEEDGCSCRFPEEEEGGCELRAKRSGSGATNFS
LLKQAGDVEENPGPMVSKGEEDNMAIIKEFMRFKVHMEGSVNGHEFEIEGEGEGRP
YEGTQTAKLKVTKGGPLPFAWDILSPQFMYGSKAYVKHPADIPDYLKLSFPEGFKW
ERVMNFEDGGVVTVTQDSSLQDGEFIYKVKLRGTNFPSDGPVMQKKTMGWEASSE
RMYPEDGALKGEIKQRLKLKDGGHYDAEVKTTYKAKKPVQLPGAYNVNIKLDITSH
NEDYTIVEQYERAEGRHSTGGMDELYKGTGAGSG
SEQ ID NO: 18
ATGAAGTGGAAGGCGCTTTTCACCGCGGCCATCCTGCAGGCACAGTTGCCGATTA
CAGAGGCACAGAGCTTTGGCCTGCTGGATCCCAAACTCTGCTACCTGCTGGATGG
AATCCTCTTCATCTATGGTGTCATTCTCACTGCCTTGTTCCTGAGAGTGAAGTTCA
GCAGGAGCGCAGACGCCCCCGCGTACCAGCAGGGCCAGAACCAGCTCTATAACG
AGCTCAATCTAGGACGAAGAGAGGAGTACGATGTTTTGGACAAGAGACGTGGCC
GGGACCCTGAGATGGGGGGAAAGCCGCAGAGAAGGAAGAACCCTCAGGAAGGC
CTGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTCTC
AGTACAGCCACCAAGGACACCTACGACGCCCTTCACATGCAGGCCCTGCCCCCTC
GCGGTCTCAGTACAGCCACCAAGGACACCTACGACGCCCTTCACATGCAGGCCC
TGCCCCCTCGCGGATCGGGTGGGACTAGTGGCaaacgcggccgcaaaaaactgctgtatatttttaaaca
gccgtttatgcgcccggtgcagaccacccaggaagaagatggctgcagctgccgctttccggaagaagaagaaggcggctgcgaa
ctgCGGGCCAAGCGGTCCGGATCCGGAGCCACCAACTTCAGCCTGCTGAAGCAGG
CCGGCGACGTGGAGGAGAACCCCGGCCCCATGgtgagcaagggcgaggaggataacatggccatca
tcaaggagttcatgcgcttcaaggtgcacatggagggctccgtgaacggccacgagttcgagatcgagggcgagggcgagggccg
cccctacgagggcacccagaccgccaagctgaaggtgaccaagggtggccccctgcccttcgcctgggacatcctgtcccctcagtt
catgtacggctccaaggcctacgtgaagcaccccgccgacatccccgactacttgaagctgtccttccccgagggcttcaagtggga
gcgcgtgatgaacttcgaggacggcggcgtggtgaccgtgacccaggactcctccctgcaggacggcgagttcatctacaaggtga
agctgcgcggcaccaacttcccctccgacggccccgtaatgcagaagaagaccatgggctgggaggcctcctccgagcggatgta
ccccgaggacggcgccctgaagggcgagatcaagcagaggctgaagctgaaggacggcggccactacgacgctgaggtcaaga
ccacctacaaggccaagaagcccgtgcagctgcccggcgcctacaacgtcaacatcaagttggacatcacctcccacaacgaggac
tacaccatcgtggaacagtacgaacgcgccgagggccgccactccaccggcggcatggacgagctgtacaagggaaccggtGC
TggaagtggtTAA
SEQ ID NO: 19
MKWKALFTAAILQAQLPITEAQSFGLLDPKLCYLLDGILFIYGVILTALFLRVKFSRSA
DAPAYQQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPRGSGGT
SGKRGRKKLLYIFKQPFMRPVQTTQEEDGCSCRFPEEEEGGCELRAKRSGSGATNFS
LLKQAGDVEENPGPMVSKGEEDNMAIIKEFMRFKVHMEGSVNGHEFEIEGEGEGRP
YEGTQTAKLKVTKGGPLPFAWDILSPQFMYGSKAYVKHPADIPDYLKLSFPEGFKW
ERVMNFEDGGVVTVTQDSSLQDGEFIYKVKLRGTNFPSDGPVMQKKTMGWEASSE
RMYPEDGALKGEIKQRLKLKDGGHYDAEVKTTYKAKKPVQLPGAYNVNIKLDITSH
NEDYTIVEQYERAEGRHSTGGMDELYKGTGAGSG

1-18. (canceled)

19. A method of modifying an endogenous cell surface protein gene locus in a human T cell, comprising:
(a) introducing into the human T cell
(i) a targeted nuclease that cleaves a target region encoding the N-terminus of the endogenous cell surface protein gene locus to create an insertion site in the genome of the cell; and
(ii) a heterologous nucleic acid sequence comprising a coding or a non-coding sequence, wherein the nucleic acid sequence is flanked by homologous sequences, and
(b) allowing homologous recombination to take place, thereby inserting the heterologous nucleic acid sequence in the insertion site to generate a human T cell comprising a modified endogenous cell surface protein gene locus.

20. The method of claim 19, wherein the heterologous nucleic acid sequence comprises a non-coding sequence, and wherein the heterologous nucleic acid sequence is inserted into the 5′ non-coding sequence of the endogenous cell surface protein gene locus.

21. The method of claim 20, wherein the non-coding sequence comprises an exogenous regulatory sequence and wherein, upon insertion of the exogenous regulatory sequence in the 5′ non-coding sequence, the endogenous cell surface protein is expressed under the regulatory control of the exogenous regulatory sequence.

22. The method of claim 21, wherein the exogenous regulatory sequence is a promoter.

23. The method of claim 19, wherein the heterologous nucleic acid sequence is inserted into the coding region of the cell surface protein gene locus, wherein the heterologous nucleic acid sequence comprises a coding sequence, and wherein, upon insertion, the heterologous nucleic acid is under the control of an endogenous regulatory sequence in the endogenous cell surface protein gene locus.

24. The method of claim 23, wherein the heterologous nucleic acid comprises, in the following order, a coding sequence and a poly A sequence.

25. The method of claim 23, wherein the heterologous nucleic acid sequence comprises, in the following order, a coding sequence and a self-cleaving peptide sequence.

26. The method of claim 19, wherein the targeted nuclease introduces a double-stranded break at the insertion site.

27. The method of claim 19, wherein the targeted nuclease is an RNA-guided nuclease.

28. The method of claim 27, wherein the RNA-guided nuclease is a Cpf1 nuclease or a Cas9 nuclease and the method further comprises introducing into the cell a guide RNA that specifically hybridizes to the target region.

29. The method of claim 28, wherein the Cpf1 nuclease or the Cas9 nuclease, the guide RNA and the nucleic acid are introduced into the cell as a ribonucleoprotein complex (RNP)-nucleic acid sequence complex, wherein the RNP-nucleic acid sequence complex comprises:
(i) the RNP, wherein the RNP comprises the Cpf1 nuclease or the Cas9 nuclease and the guide RNA; and
(ii) the nucleic acid sequence.

30. The method of claim 19, wherein the T cell is a primary T cell.

31. The method of claim 30, wherein the primary T cell is a regulatory T cell.

32. The method of claim 30, wherein the primary T cell is a CD8+ T cell or a CD4+ T cell.

33. The method of claim 32, wherein the primary T cell is a CD4+CD8+ T cell.

34. The method of claim 19, further comprising culturing the modified T cells under conditions effective for expanding the population of modified cells.

35. The method of claim 19, further comprising purifying T cells that express the modified endogenous cell surface protein.

36. A modified human T cell produced by any one of the methods of claim 19.

37. A method of enhancing an immune response in a human subject comprising:
a) obtaining T cells from the subject;
b) modifying the T cells using the method of claim 19; and
c) administering the modified T cells to the subject.

38-62. (canceled)

63. The method of claim 19, wherein the endogenous cell surface protein is selected from the group consisting of a T cell receptor (TCR) complex protein, a co-stimulatory receptor, a co-inhibitory receptor, a cytokine receptor and a chemokine receptor.

64. The method of claim 63, wherein the TCR complex protein is selected from the group consisting of: the TCR-α chain, the TCR-β chain, the CD3δ chain, the CD3ε chain, the CD3γ chain, and the CD3ζ chain of the endogenous TCR complex.
